(19) United States
(12) Patent Application Publication
(10) Pub. No.: US 2007/0072812 A1
(43) Pub. Date: Mar. 29, 2007

(54) Quaternary salt derivatives of 1,4-diphenylazetidin-2-ones

(75) Inventors: Eduardo J. Martinez, New York, NY (US); John J. Talley, Somerville, MA (US)

Correspondence Address: HESLIN ROTHENBERG FARLEY & MESITI PC, 5 COLUMBIA CIRCLE, ALBANY, NY, 12203, US

(73) Assignee: Microbia, Inc., Cambridge, MA (US)

(21) Appl. No.: 10/569,557

(22) PCT Filed: Aug. 25, 2004
(86) PCT No.: PCT/US04/27540

§ 371 Date: Oct. 27, 2006

Related U.S. Application Data
(60) Provisional application No. 60/497,590, filed on Aug. 25, 2003.

(51) Int. Cl. A61K 31/7052; A61K 31/498; A61K 31/397; C07D 403/14

(52) U.S. Cl. 514/23; 514/210.02; 540/200; 536/17.4

(57) ABSTRACT

Quaternary salt derivatives of 1,4-diphenylazetidin-2-ones useful for the treatment of hypercholesterolemia are disclosed. The compounds are of the general formulae 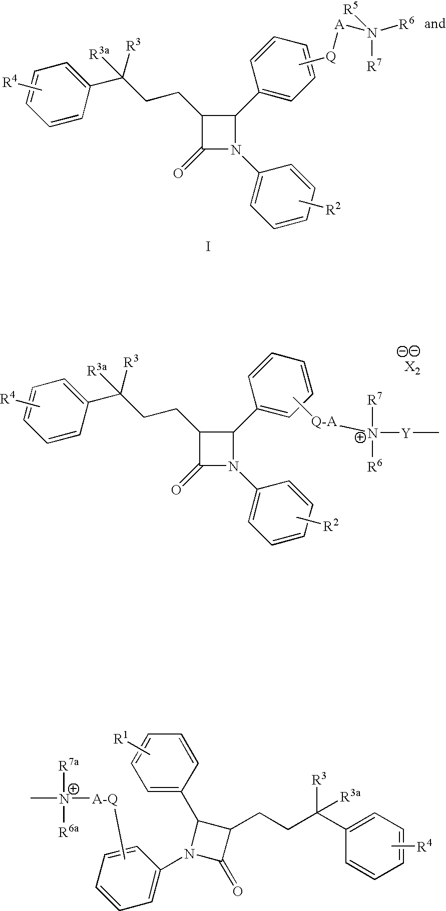 as well as isomers of these formulae.

### FIELD OF THE INVENTION

The invention relates to a chemical genus of quaternary salt derivatives of 1,4-diphenylazetidin-2-ones useful for the treatment of hypercholesterolemia.

### BACKGROUND OF THE INVENTION

1,4-Diphenylazetidin-2-ones and their utility for treating disorders of lipid metabolism are described in U.S. Pat. No. 6,498,156, USRE37721 and PCT application WO02/50027, the disclosures of which are incorporated herein by reference as they relate to utility.

### SUMMARY OF THE INVENTION

In one aspect the invention relates to compounds of the general formulae: 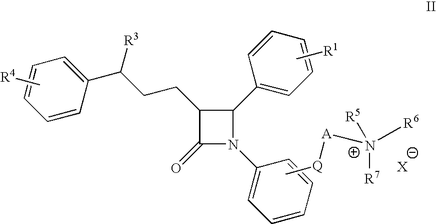
wherein
R¹ and R² are chosen from H, halogen, —OH, loweralkyl, —O-loweralkyl, —CN, —-S-loweralkyl, amino, acyl, lower aminoalkyl, alkylsulfonyl, arylsulfonyl, a sugar, a glucuronide and a sugar carbamate;
R³ is chosen from H, —OH, fluoro and —O-loweralkyl;
R^{3a} is chosen from H and fluoro, or R^{3a} and R³ together are ═O;
R⁴ is chosen from H, halogen, —OH, loweralkyl, —O-loweralkyl, —CN, —S-loweralkyl, amino, acyl and lower aminoalkyl, alkylsulfonyl, arylsulfonyl;
Q is chosen from a direct bond, —O—, —S—, —NH—, —CH₂O—, —CH₂NH—, —C(═O)—, —CONH—, —NHCO—, —O(C═O)—, —(C═O)O—, —NHCONH—, —OCONH—and —NHCOO—;
A is chosen from C₂ to C₂₀ hydrocarbon, substituted alkyl of 2 to 20 carbons, substituted aryl, substituted arylalkyl, and oxaalkyl of four to fifty carbons; and, when Q is a direct bond, —C(═O) or —O(C═O)—, A may additionally be methylene;
R⁵ forms a five- to seven-membered ring with A or R⁶;
R⁶ is alkyl, forms a double bond with A or forms a five- to seven-membered ring with R⁵;
R⁷ is alkyl or together with R¹or R⁶ forms a second five- to seven-membered ring; and when Q is not —O— or CH₂NH—, R⁵, may additionally be alkyl or aryl; and
X is an anion.

In a second aspect the invention relates to compounds that may be thought of as isomeric “dimers” of the foregoing quats, namely isomers of formulae III, IV and V: 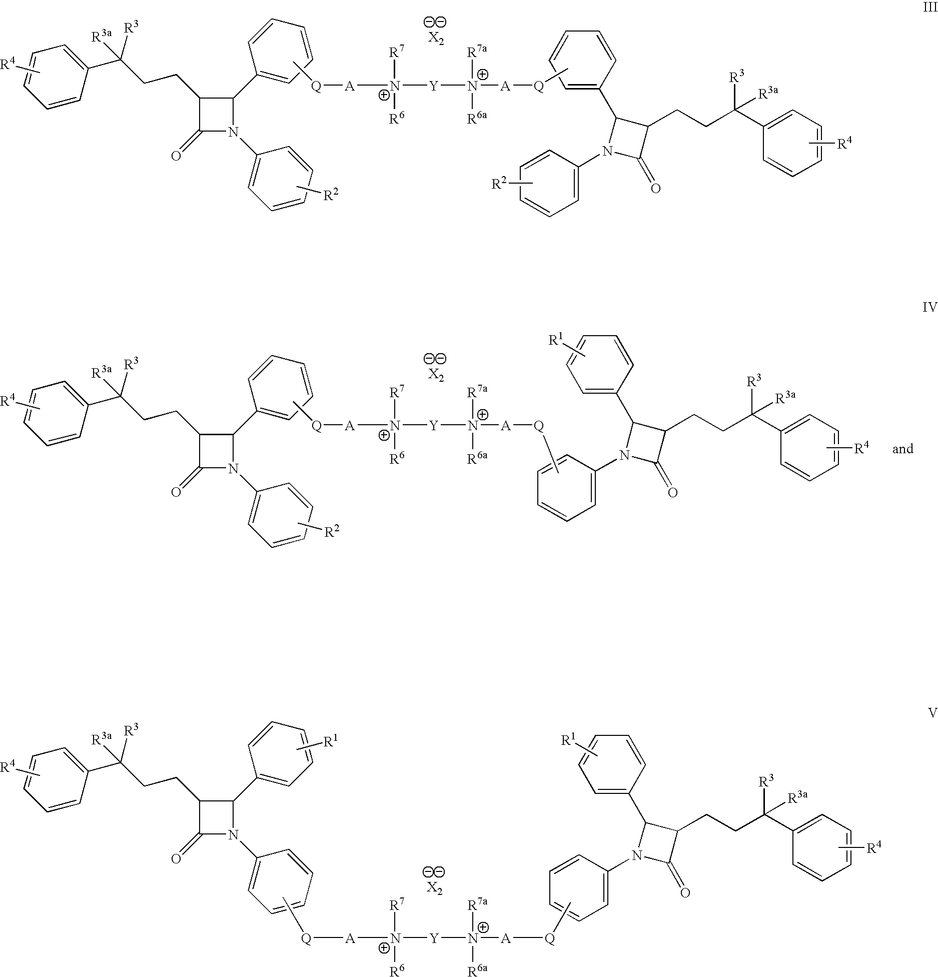
in which the substituents are as defined before, and Y is chosen from C₂ to C₂₀ hydrocarbon, substituted alkyl of 2 to 20 carbons, substituted arylalkyl and oxaalkyl of four to fifty carbons;
R⁶ and R⁶a are alkyl or together with Y form a first five- to seven-membered ring;
R⁷ and R⁷a are alkyl or together form a second five- to seven-membered ring; and
X₂ is either a dianion or two monoanions.

In a third aspect the invention relates to pharmaceutical formulations comprising a pharmaceutically acceptable carrier and a compound as above having a pharmaceutically acceptable counter anion and, optionally additionally comprising one or more of (1) an inhibitor of cholesterol biosynthesis; (2) a cholesterol ester transfer protein (CETP) inhibitor; (3) a bile acid sequestrant; (4) a nicotinic acid or derivative thereof; (5) a peroxisome proliferator-activated receptor activator; (6) an acylcoenzyme A:cholesterol acyltransferase (ACAT) inhibitor; (7) an obesity control medication; and (8) a compound that normalizes lipid metabolism.

In a fourth aspect, the invention relates to methods for treating a disorder of lipid metabolism, including hyperlipidemia, sitosterolemia and arteriosclerotic symptoms; inhibiting the absorption of cholesterol from the intestine; reducing the blood plasma or serum concentrations of LDL cholesterol; reducing the concentrations of cholesterol and cholesterol ester in the blood plasma or serum; reducing blood plasma or serum concentrations of C-reactive protein (CRP), reducing blood plasma or serum concentrations of triglycerides; reducing blood plasma or serum concentrations of apolipoprotein B; increasing blood plasma or serum concentrations of high density lipoprotein (HDL) cholesterol; increasing the fecal excretion of cholesterol; treating a clinical condition for which a cholesterol absorption inhibitor is indicated and reducing the incidence of coronary heart disease-related events; reducing plasma or tissue concentration of at least one non-cholesterol sterol or 5α-stanol; treating or preventing vascular inflammation; preventing, treating, or ameliorating symptoms of Alzheimer's Disease; regulating the production or level of at least one amyloid β peptide in bloodstream and/or brain of a subject; regulating the amount of ApoE isoform 4 in the bloodstream and/or brain; preventing and/or treating obesity; and preventing or decreasing the incidence of xanthomas. The methods comprise administering a compound described herein.

In a fifth aspect, the invention relates to methods and compositions for prevention or treatment of a cholesterol-associated tumor. The methods comprise administering a therapeutically effective amount of a compound of the invention to a patient at risk of developing a cholesterol-associated tumor or already exhibiting a cholesterol-associated tumor. The method also includes coadministering a therapeutically effective amount of a compound of the invention and at least one other anticancer agent.

In a sixth aspect, the invention relates to an article of manufacture comprising a container, instructions, and a pharmaceutical formulation as described above. The instructions are for the administration of the pharmaceutical formulation for a purpose chosen from: the prevention or treatment of a disorder of lipid metabolism; inhibiting the absorption of cholesterol from the intestine; reducing the plasma or tissue concentration of at least one non-cholesterol sterol or 5a-stanol; reducing the blood plasma or serum concentrations of LDL cholesterol; reducing the concentrations of cholesterol and cholesterol ester in the blood plasma or serum; increasing the fecal excretion of cholesterol; reducing the incidence of coronary heart disease-related events; reducing blood plasma or serum concentrations of C-reactive protein (CRP); treating or preventing vascular inflammation; reducing blood plasma or serum concentrations of triglycerides; increasing blood plasma or serum concentrations of HDL cholesterol; reducing blood plasma or serum concentrations of apolipoprotein B; preventing, treating, or ameliorating symptoms of Alzheimer's Disease; regulating the production of amyloid β peptide; regulating the amount of ApoE isoform 4 in the bloodstream and/or brain; preventing and/or treating obesity; preventing or decreasing the incidence of xanthomas; and preventing or treating a cholesterol-associated tumor.

### DETAILED DESCRIPTION OF THE INVENTION

Compounds of the genera I-V above are inhibitors of cholesterol absorption from the intestine. As such they have utility in treating and preventing lipid disorders, such as hypercholesterolemia and hyperlipidemia. Because of their effect in lowering serum lipids, the compounds are useful in the treatment and prevention of atherosclerosis. The compounds can be used advantageously in combination with other hypolipidemic agents, including inhibitors of cholesterol biosynthesis, such as the HMG-CoA reductase inhibitors. Preferred HMG-CoA reductase inhibitors would include the “statins”: lovastatin, simvastatin, pravastatin, rosuvastatin, mevastatin, atorvastatin, cerivastatin, pitavastatin and fluvastatin. A further listing of non-limiting examples of antihyperlipidemic agents that may be used in combination with the compounds of the present invention may be found in columns 5-6 of U.S. Pat. No. 6,498,156, the disclosure of which is incorporated herein by reference.

As described above, the formulation may additionally contain at least one bile acid sequestrant. Sequestrants include cholestyramine, colestipol and colesevelam hydrochloride. The formulation may also contain a nicotinic acid or derivative thereof. Nicotinic acid derivatives include niceritrol, nicofuranose and acipimox. The formulation may also contain a peroxisome proliferator-activated receptor activator, which may be a fibric acid derivative. Fibric acids include fenofibrate, clofibrate, gemfibrozil, ciprofibrate, bezafibrate, clinofibrate, binifibrate and lifibrol. The formulation may also contain a CETP inhibitor. Examples of such are the compounds identified as JTT-705 in Nature. 406, (6792):203-7 (2000 ) and CP-529,414 (torcetrapib), described in US20030186952 and WO2000017164. Examples of CETP inhibitors are also found in Current Opinion in Investigational Drugs. 4(3):291-297 (2003). The formulation may also contain an ACAT inhibitor. Examples of such are the compounds identified as avasimibe in Current Opinion in Investigational Drugs. 3(9):291-297 (2003), and CL-277,082 in Clin Pharmacol Ther. 48(2):189-94 (1990). The formulation may also contain an obesity control medication. Examples of obesity control medications include gut hormone fragment peptide YY₃₋₃₆ (PYY₃₋₃₆)(N. Engl. J Med. 349:941, 2003; IKPEAPGE DASPEELNRY YASLRHYLNL VTRQRY) or a variant thereof, glp-1 (glucagon-like peptide-1), exendin-4 (an inhibitor of glp-1), sibutramine, phentermine, phendimetrazine, benzphetamine hydrochloride (Didrex), orlistat (Xenical), diethylpropion hydrochloride (Tenuate), fluoxetine (Prozac), bupropion, ephedra, chromium, garcinia cambogia, benzocaine, bladderwrack (focus vesiculosus), chitosan, nomame herba, galega (Goat's Rue, French Lilac), conjugated linoleic acid, L-carnitine, fiber (psyllium, plantago, guar fiber), caffeine, dehydroepiandrosterone, germander (teucrium chamaedrys), B-hydroxy-β-methylbutyrate, ATL-962 (Alizyme PLC), and T71 (Tularik, Inc.; Boulder CO), a ghrelin antagonist, Acomplia (rimonabant), AOD9604, alpha-lipoic acid (alpha-LA), and pyruvate.

The present invention is also directed to methods of prevention or treatment of a cholesterol-associated tumor in patients who are either at risk of developing a cholesterol-associated tumor or already exhibit a cholesterol-associated tumor. The tumor may be either a benign or a malignant tumor of the prostate, breast, endometrium or colon. The compounds of the invention may be co-administered with at least one other anticancer agent, which may be a steroidal antiandrogen, a non-steroidal antiandrogen, an estrogen, diethylstilbestrol, a conjugated estrogen, a selective estrogen receptor modulator (SERM), a taxane, or an LHRH analog. Tests showing the efficacy of the therapy and the rationale for combination therapy are presented in PCT application WO 2004/010948, the disclosure of which is incorporated herein by reference.

The compounds of the invention may reduce both cholesterol levels in vivo and epoxycholesterol formation and thereby inhibit initiation and progression of benign and malignant cholesterol-associated tumors or cholesterol-associated cell growth or cell-masses. Compositions disclosed herein, for example, are useful for the treatment and/or prevention of benign prostatic hypertrophy, as well as tumors associated with prostate, colon, endometrial, or breast tissues.

Compositions of the invention comprise an effective dose or a pharmaceutically effective amount or a therapeutically effective amount of a compound described above and may additionally comprise at least one other anticancer agent, for the treatment or prevention of benign prostatic hypertrophy or other cholesterol-related benign or malignant tumors, particularly those associated with prostate, breast, endometrial or colon tissues. Examples of agents for use in compositions and methods of the invention include steroidal or non steroidal antiandrogens such as finasteride (PROSCAR®), cyproterone acetate (CPA), flutamide (4′-nitro-3′-trifluorormethyl isobutyranilide), bicalutamide (CASODEX®), and nilutamide; estrogens, diethylstilbestrol (DES); conjugated estrogens (e.g., PREMARIN®); selective estrogen receptor modulator (SERM) compounds such as tamoxifen, raloxifene, droloxifene, idoxifene; taxanes such as paclitaxel (TAXOL®) and docetaxel (TAXOTERE®); and LHRH analogs such as goserelin acetate (ZOLADEX®), and leuprolide acetate (LUPRON®).

Methods of the invention parallel the compositions and formulations. The methods comprise co-administering to a patient in need of treatment a therapeutically effective amount of an azetidinone according to the invention and one or more of: (a) a steroidal or non steroidal antiandrogen; (b) an estrogen; (c) diethylstilbestrol (DES); (d) a conjugated estrogen; (e) a selective estrogen receptor modulator (SERM), (f) a taxane; and (g) an LHRH analog. The term “selective estrogen receptor modulator” includes both estrogen agonist and estrogen antagonists and refers to compounds that bind with the estrogen receptor, inhibit bone turnover and prevent bone loss. In particular, estrogen agonists are compounds capable of binding to the estrogen receptor sites in mammalian tissue and mimicking the actions of estrogen in that tissue. Estrogen antagonists are compounds capable of binding to the estrogen receptor sites in mammalian tissue and blocking the actions of estrogen in that tissue. Exemplary SERMs are: tamoxifen (U.S. Pat. No. 4,536,516); 4-hydroxytamoxifen (U.S. Pat. No. 4,623,660); raloxifene (U.S. Pat. No. 4,418,068); idoxifene (U.S. Pat. No. 4,839,155; and droloxifene. For the taxanes see U.S. Pat. Nos. 6,395,770; 6,380,405; and 6,239,167. Azetidinones of the invention may also be combined with a steroidal or non steroidal antiandrogen, as described above.

Certain compounds of the invention have the advantage that they suppress serum cholesterol and/or LDL levels but the compounds themselves are not appreciably absorbed into the mammalian circulation upon oral administration. As a result of the low-to-insignificant serum levels, fewer side-effects, such as drug-drug interactions, are observed.

Within the genus of the invention, subgenera include (a) those in which R⁷ forms a second six-membered ring; (b) those in which -Q-A- is chosen from (C₂ to C₂₀ hydrocarbon), —O—(C₂ to C₂₀ hydrocarbon), —NH(C₂ to C₂₀ hydrocarbon), —NHCO(C₂ to C₂₀ hydrocarbon) and oxaalkyl of four to fifty carbons; (c) those in which R¹ and R² are H, halogen, —OH, or methoxy; R³ is —OH; and R⁴ is fluoro; (d) those in which R¹ and R² are chosen from a sugar, a glucuronide and a sugar carbamate; R³ is —OH; and R⁴ is fluoro;
and (e) those in which -Q-A- is

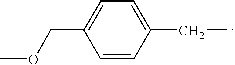

One preferred subgenus of genera I and II is that in which R⁵,R⁶ and R⁷ talen together form a diazabicyclooctane quat:

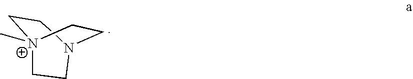

A subgenus of genera II, IV and V is that in which R⁶ and R⁶l taken together with Y form a dialkyl piperazinium bisquat:

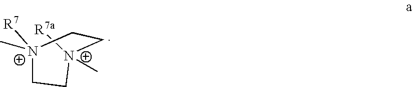

A further subgenus of genera HI, IV and V related to the dialkyl piperazinium bisquats is that in which R⁶, R⁶a, R⁷ and R7a taken together with Y form a diazabicyclooctane bisquat:

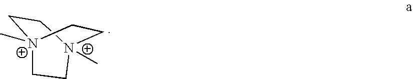

Another preferred subgenus of genera I and II is that in which R⁵,R⁶ and R⁷ taken together form a quinuclidinium quat:

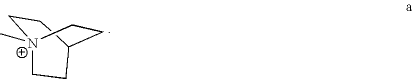

Exemplary compounds of the invention include:

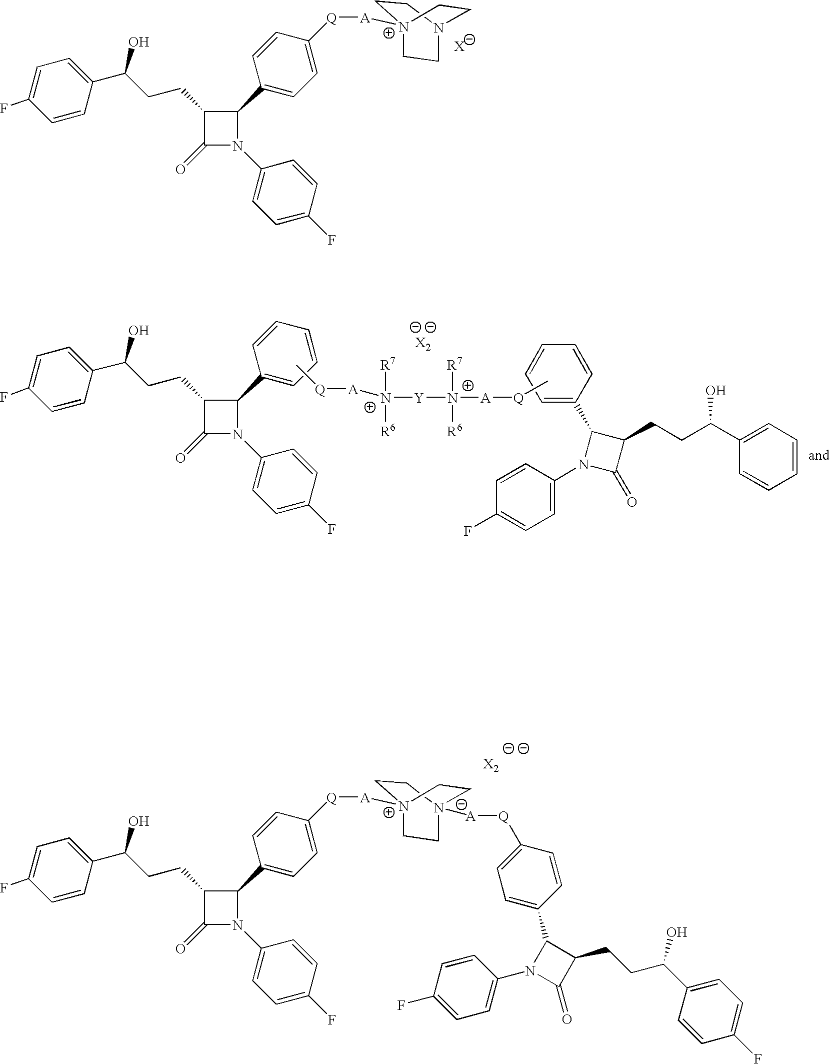

Other subgenera of genera I and II are those in which R⁵ forms a five- to seven-membered ring with A, R⁶ forms a double bond with A and R⁷ is alkyl.

The compounds of the invention are quaternary salts, i.e. cationic species. Therefore they will always be presented as salts, and the term “pharmaceutically acceptable salt” refers to salts whose counter ion (anion) derives from pharmaceutically acceptable non-toxic acids including inorganic acids, organic acids and water (which formally furnishes the hydroxide anion). Suitable pharmaceutically acceptable anions for the compounds of the present invention include hydroxide, acetate, benzenesulfonate (besylate), benzoate, bicarbonate, bisulfate, carbonate, camphorsulfonate, citrate, ethanesulfonate, fiunarate, gluconate, glutamate, glycolate, bromide, chloride, isethionate, lactate, maleate, malate, mandelate, methanesulfonate, mucate, nitrate, pamoate, pantothenate, phosphate, succinate, sulfate, tartrate, trifluoroacetate, p-toluenesulfonate, acetamidobenzoate, adipate, alginate, aminosalicylate, anhydromethylenecitrate, ascorbate, aspartate, calcium edetate, camphorate, camsylate, caprate, caproate, caprylate, cinnarnate, cyclamate, dichloroacetate, edetate (EDTA), edisylate, embonate, estolate, esylate, fluoride, formate, gentisate, gluceptate, glucuronate, glycerophosphate, glycolate, glycollylarsanilate, hexylresorcinate, hippurate, hydroxynaphthoate, iodide, lactobionate, malonate, mesylate, napadisylate, napsylate, nicotinate, oleate, orotate, oxalate, oxoglutarate, palmitate, pectinate, pectinate polymer, phenylethylbarbiturate, picrate, pidolate, propionate, rhodanide, salicylate, sebacate, stearate, tannate, theoclate, tosylate and the like. The desired salt may be obtained by ion exchange of whatever counter ion is obtained in the synthesis of the quat. These methods are well known to persons of skill. Although pharmaceutically acceptable counter ions will be preferred for preparing pharmaceutical formulations, other anions are quite acceptable as synthetic intermediates. Thus X may be pharmaceutically undesirable anions, such as iodide, oxalate, trifluoromethanesulfonate and the like, when such salts are chemical intermediates. When the compounds of the invention are bisquats, one may employ as counter ions either two monoanionic species (e.g. Cl₂) or a single dianionic species (e.g. fumarate). Similarly, one could employ oligoanionic species and make salts having appropriate ratios of quat to counterion, such as (quat)₃ citrates. These would be obvious equivalents.

### DEFINITIONS

Throughout this specification the terms and substituents retain their definitions.

Alkyl is intended to include linear, branched, or cyclic hydrocarbon structures and combinations thereof. Lower alkyl refers to alkyl groups of from 1 to 6 carbon atoms. Examples of lower alkyl groups include methyl, ethyl, propyl, isopropyl, butyl, s-and t-butyl and the like. Preferred alkyl groups are those of C₂₀ or below. Cycloalkyl is a subset of alkyl and includes cyclic hydrocarbon groups of from 3 to 8 carbon atoms. Examples of cycloalkyl groups include c-propyl, c-butyl, c-pentyl, norbornyl, adamantyl and the like.

C₁ to C₂₀ Hydrocarbon includes alkyl, cycloalkyl, alkenyl, alkynyl, aryl and combinations thereof. Examples include phenethyl, cyclohexylmethyl, camphoryl and naphthylethyl.

Alkoxy or alkoxyl refers to groups of from 1 to 8 carbon atoms of a straight, branched, cyclic configuration and combinations thereof attached to the parent structure through an oxygen. Examples include methoxy, ethoxy, propoxy, isopropoxy, cyclopropyloxy, cyclohexyloxy and the like. Lower-alkoxy refers to groups containing one to four carbons.

Oxaalkyl refers to alkyl residues in which one or more carbons (and their associated hydrogens) have been replaced by oxygen. Examples include methoxypropoxy, 3,6,9-trioxadecyl and the like. The term oxaalllyl is intended as it is understood in the art [see Naming and Indexing of Chemical Substances for Chemical Abstracts, published by the American Chemical Society, ¶196, but without the restriction of ¶127(a)], i.e. it refers to compounds in which the oxygen is bonded via a single bond to its adjacent atoms (forming ether bonds). Similarly, thiaalkyl and azaalkyl refer to alkyl residues in which one or more carbons have been replaced by sulfur or nitrogen, respectively. Examples include ethylaminoethyl and methylthiopropyl.

Acyl refers to groups of from 1 to 8 carbon atoms of a straight, branched, cyclic configuration, saturated, unsaturated and aromatic and combinations thereof, attached to the parent structure through a carbonyl functionality. One or more carbons in the acyl residue may be replaced by nitrogen, oxygen or sulfur as long as the point of attachment to the parent remains at the carbonyl. Examples include acetyl, benzoyl, propionyl, isobutyryl, t-butoxycarbonyl, benzyloxycarbonyl and the like. Lower-acyl refers to groups containing one to four carbons.

Aryl and heteroaryl mean a 5- or 6-membered aromatic or heteroaromatic ring containing 0-3 heteroatoms selected from O, N, or S; a bicyclic 9- or 10-membered aromatic or heteroaromatic ring system containing 0-3 heteroatoms selected from O, N, or S; or a tricyclic 13- or 14-membered aromatic or heteroaromatic ring system containing 0-3 heteroatoms selected from O, N, or S. Aromatic 6- to 14-membered carbocyclic rings include, e.g., benzene, naphthalene, indane, tetralin, and fluorene and the 5- to 10-membered aromatic heterocyclic rings include, e.g., imidazole, pyridine, indole, thiophene, benzopyranone, thiazole, furan, benzimidazole, quinoline, isoquinoline, quinoxaline, pyrimidine, pyrazine, tetrazole and pyrazole.

Arylalkyl means an alkyl residue attached to an aryl ring. Examples are benzyl, phenethyl and the like.

Substituted alkyl, aryl, cycloalkyl, heterocyclyl etc. refer to alkyl, aryl, cycloalkyl, or heterocyclyl wherein up to three H atoms in each residue are replaced with halogen, haloalkyl, hydroxy, loweralkoxy, carboxy, carboalkoxy (also referred to as allcoxycarbonyl), carboxamido (also referred to as alkylaminocarbonyl), cyano, carbonyl, nitro, amino, alkylamino, dialkylamino, mercapto, alkylthio, sulfoxide, sulfone, acylamino, amidino, phenyl, benzyl, heteroaryl, phenoxy, benzyloxy, or heteroaryloxy.

The term “halogen” means fluorine, chlorine, bromine or iodine.

The term “sugar” is used in its normal sense, as defined in Hawley's Condensed Chemical Dictionar 12^{th} Edition, Richard J. Lewis, Sr.; Van Nostrand Reinhold Co. New York. It encompasses any carbohydrate comprised of one or two saccharose groups. The monosaccharide sugars (often called simple sugars) are composed of chains of 2-7 carbon atoms. One of the carbons carries aldehydic or ketonic oxygen, which may be combined in acetal or ketal forms. The remaining carbons usually have hydrogen atoms and hydroxyl groups. Among monosaccharides which would be considered within the term “sugars” as intended in this application, are arabinose, ribose, xylose, ribulose, xylulose, deoxyribose, galactose, glucose, mannose, fructose, sorbose, tagatose, fucose, quinovose, rhamnose, manno-heptulose and sedoheptulose. Among the disaccharides are sucrose, lactose, maltose, and cellobiose. Unless specifically modified, the general term “sugar” refers to both D-sugars and L-sugars.

The term “glucuronide” is also used in its normal sense to refer to a glycoside of glucuronic acid.

The term “sugar carbamate” refers to mono-, di- and oligosaccharides in which one or more hydroxyls have been derivatized as carbamates, particularly as phenyl carbamates and substituted phenyl carbamates. [See Detmers et al. Biochim Biophvs. Acta 1486, 243-252 (2000), which is incorporated herein by reference.] A preferred sugar carbamate is:

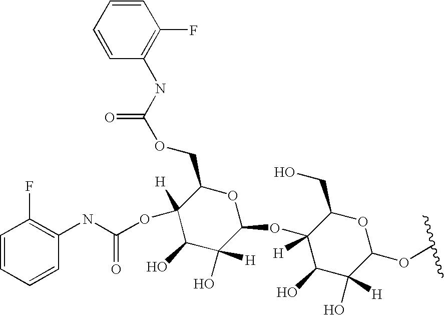

The term “prodrug” refers to a compound that is made more active in vivo. Since the compounds of the invention are minimally absorbed into the systemic circulation, activation in vivo may come about by chemical action or through the intermediacy of enzymes and microflora in the GI tract.

In the characterization of the variables, it is recited that R⁵ may form a five- to seven-membered ring with A or R⁶; that R⁶ may form a double bond with A or may form a five- to seven-membered ring with R⁵; and that R⁷ may form a second five- to seven-membered ring. It is intended that these rings may exhibit various degrees of unsaturation (from fully saturated to aromatic), may include heteroatoms and may be substituted with lower alkyl or alkoxy. Examples of the -A-NR⁵R⁶R⁷ residue that fall within this subgenus include:

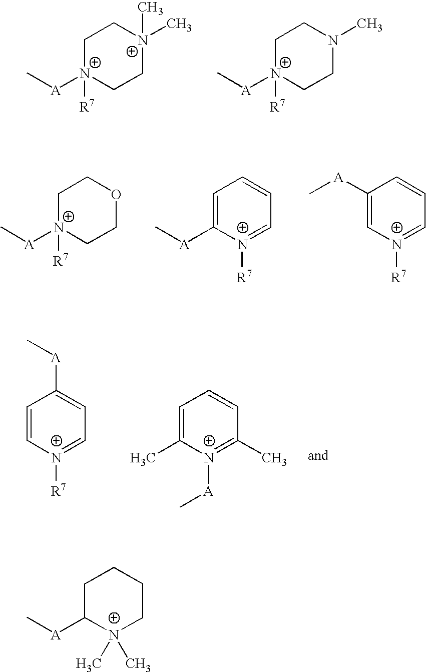

It will be recognized that the compounds of this invention can exist in radiolabeled form, i.e., the compounds may contain one or more atoms containing an atomic mass or mass number different from the atomic mass or mass number usually found in nature. Radioisotopes of hydrogen, carbon, phosphorous, fluorine, and chlorine include ³H, ¹⁴C, ³⁵S, ¹⁸F, and ³⁶Cl, respectively. Compounds that contain those radioisotopes and/or other radioisotopes of other atoms are within the scope of this invention. Tritiated, i.e. ³H, and carbon-14, i.e., ¹⁴C, radioisotopes are particularly preferred for their ease in preparation and detectability. Radiolabeled compounds of Formulas I-VII of this invention and prodrugs thereof can generally be prepared by methods well known to those skilled in the art. Conveniently, such radiolabeled compounds can be prepared by carrying out the procedures disclosed in the Examples and Schemes by substituting a readily available radiolabeled reagent for a non-radiolabeled reagent.

The terms “methods of treating or preventing” mean amelioration, prevention or relief from the symptoms and/or effects associated with lipid disorders. The term “preventing” as used herein refers to administering a medicament beforehand to forestall or obtund an acute episode. The person of ordinary skill in the medical art (to which the present method claims are directed) recognizes that the term “prevent” is not an absolute term. In the medical art it is understood to refer to the prophylactic administration of a drug to substantially diminish the likelihood or seriousness of a condition, and this is the sense intended in applicants' claims. As used herein, reference to “treatment” of a patient is intended to include prophylaxis. Throughout this application, various references are referred to within parentheses or square brackets. The disclosures of these publications in their entireties are hereby incorporated by reference as if written herein.

The term “mammal” is used in its dictionary sense. The term “mammal” includes, for example, mice, hamsters, rats, cows, sheep, pigs, goats, and horses, monkeys, dogs (e.g., Canis familiaris), cats, rabbits, guinea pigs, and primates, including humans.

The compounds may be used to treat or prevent vascular inflammation, as described in US published application 20030119757; to prevent, treat, or ameliorate symptoms of Alzheimer's Disease and to regulate the production or level of amyloid β peptide and ApoE isoform 4, as described in U.S. Pat. No. 6,080,778 and US published application 20030013699; and to prevent or decrease the incidence of xanthomas, as described in US published application 20030119809. The disclosures of all are incorporated herein by reference as they relate to utility.

The compounds described herein contain two or more asymmetric centers and may thus give rise to enantiomers, diastereomers, and other stereoisomeric forms. Each chiral center may be defined, in terms of absolute stereochemistry, as (R)- or (S)-. The present invention is meant to include all such possible isomers, as well as, their racemic and optically pure forms. Optically active (R)- and (S)-, or (D)- and (L)-isomers may be prepared using chiral synthons or chiral reagents, or resolved using conventional techniques. When the compounds described herein contain olefinic double bonds or other centers of geometric asymmetry, and unless specified otherwise, it is intended that the compounds include both E and Z geometric isomers. Likewise, all tautomeric forms are also intended to be included.

The graphic representations of racemic, ambiscalemic and scalemic or enantiomerically pure compounds used herein are taken from Maehr J. Chem. Ed. 62, 114-120 (1985): solid and broken wedges are used to denote the absolute configuration of a chiral element; wavy lines and single thin lines indicate disavowal of any stereochemical implication which the bond it represents could generate; solid and broken bold lines are geometric descriptors indicating the relative configuration shown but denoting racemic character; and wedge outlines and dotted or broken lines denote enantiomerically pure compounds of indeterminate absolute configuration. Thus, the formula X is intended to encompass both of the pure enantiomers of that pair: 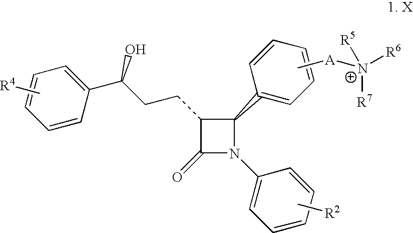
Means either pure R,S,R: 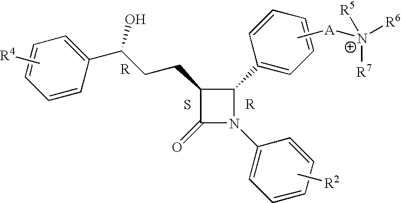
or pure S,R,S: 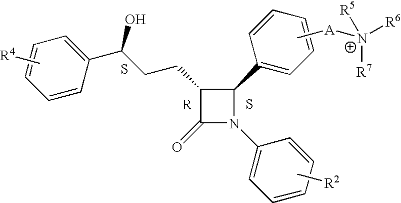
whereas 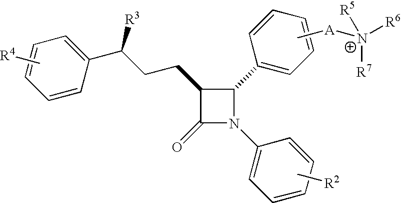
refers to a racemic mixture of S,R,S and S,S,R, i.e. having a trans relative configuration on the beta lactam ring.

The term “enantiomeric excess” is well known in the art and is defined for a

$${ee}_{a} = ( \frac{conc . of a - conc . of b}{conc . of a + conc . of b} ) \times 100$$

resolution of ab? a+b as

The term “enantiomeric excess” is related to the older term “optical purity” in that both are measures of the same phenomenon. The value of ee will be a number from 0 to 100, zero being racemic and 100 being pure, single enantiomer. A compound which in the past might have been called 98% optically pure is now more precisely described as 96% ee; in other words, a 90% ee reflects the presence of 95% of one enantiomer and 5% of the other in the material in question.

The configuration of any carbon-carbon double bond appearing herein is selected for convenience only and is not intended to designate a particular configuration; thus a carbon-carbon double bond depicted arbitrarily herein as trans may be cis, trans, or a mixture of the two in any proportion.

Terminology related to “protecting”, “deprotecting” and “protected” functionalities occurs throughout this application. Such terminology is well understood by persons of skill in the art and is used in the context of processes which involve sequential treatment with a series of reagents. In that context, a protecting group refers to a group which is used to mask a functionality during a process step in which it would otherwise react, but in which reaction is undesirable. The protecting group prevents reaction at that step, but may be subsequently removed to expose the original functionality. The removal or “deprotection” occurs after the completion of the reaction or reactions in which the functionality would interfere. Thus, when a sequence of reagents is specified, as it is in the processes of the invention, the person of ordinary skill can readily envision those groups that would be suitable as “protecting groups”. Suitable groups for that purpose are discussed in standard textbooks in the field of chemistry, such as Protective Groups in Organic Synthesis by T.W.Greene [John Wiley & Sons, New York, 1991], which is incorporated herein by reference. Particular attention is drawn to the chapters entitled “Protection for the Hydroxyl Group, Including 1,2- and 1,3-Diols” (pages 10-86).

The abbreviations Me, Et, Ph, Tf, Ts and Ms represent methyl, ethyl, phenyl, trifluoromethanesulfonyl, toluensulfonyl and methanesulfonyl respectively. A comprehensive list of abbreviations utilized by organic chemists (i.e. persons of ordinary skill in the art) appears in the first issue of each volume of the Journal of Organic Chemistry. The list, which is typically presented in a table entitled “Standard List of Abbreviations” is incorporated herein by reference.

While it may be possible for the compounds of formula (I) to be administered as the raw chemical, it is preferable to present them as a pharmaceutical composition. According to a further aspect, the present invention provides a pharmaceutical composition comprising a compound of formula (I) or a pharmaceutically acceptable salt or solvate thereof, together with one or more pharmaceutically carriers thereof and optionally one or more other therapeutic ingredients. The carrier(s) must be “acceptable” in the sense of being compatible with the other ingredients of the formulation and not deleterious to the recipient thereof.

The formulations include those suitable for oral, parenteral (including subcutaneous, intradermal, intramuscular, intravenous and intraarticular), rectal and topical (including dermal, buccal, sublingual and intraocular) administration. The most suitable route may depend upon the condition and disorder of the recipient. The formulations may conveniently be presented in unit dosage form and may be prepared by any of the methods well known in the art of pharmacy. All methods include the step of bringing into association a compound of formula (I) or a pharmaceutically acceptable salt or solvate thereof (“active ingredient”) with the carrier which constitutes one or more accessory ingredients. In general, the formulations are prepared by uniformly and intimately bringing into association the active ingredient with liquid carriers or finely divided solid carriers or both and then, if necessary, shaping the product into the desired formulation.

Formulations of the present invention suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

A tablet may be made by compression or molding, optionally with one or more accessory ingredients. Compressed tablets may be prepared by compressing in a suitable machine the active ingredient in a free-flowing form such as a powder or granules, optionally mixed with a binder, lubricant, inert diluent, lubricating, surface active or dispersing agent. Molded tablets may be made by molding in a suitable machine a mixture of the powdered compound moistened with an inert liquid diluent. The tablets may optionally be coated or scored and may be formulated so as to provide sustained, delayed or controlled release of the active ingredient therein.

The pharmaceutical compositions may include a “pharmaceutically acceptable inert carrier”, and this expression is intended to include one or more inert excipients, which include starches, polyols, granulating agents, microcrystalline cellulose, diluents, lubricants, binders, disintegrating agents, and the like. If desired, tablet dosages of the disclosed compositions may be coated by standard aqueous or nonaqueous techniques, “Pharmaceutically acceptable carrier” also encompasses controlled release means.

Compositions of the present invention may also optionally include other therapeutic ingredients, anti-caking agents, preservatives, sweetening agents, colorants, flavors, desiccants, plasticizers, dyes, and the like. Any such optional ingredient must, of course, be compatible with the compound of the invention to insure the stability of the formulation.

Examples of excipients for use as the pharmaceutically acceptable carriers and the pharmaceutically acceptable inert carriers and the aforementioned additional ingredients include, but are not limited to:

BINMERS: corn starch, potato starch, other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch (e.g., STARCH 1500® and STARCH 1500 LM®, sold by Colorcon, Ltd.), hydroxypropyl methyl cellulose, microcrystalline cellulose (e.g. AVICEL™, such as, AVICEL-PH-101™, -103™ and -105™, sold by FMC Corporation, Marcus Hook, Pa., USA), or mixtures thereof;

FILLERS: talc, calcium carbonate (e.g., granules or powder), dibasic calcium phosphate, tribasic calcium phosphate, calcium sulfate (e.g., granules or powder), microcrystalline cellulose, powdered cellulose, dextrates, kaolin, mannitol, silicic acid, sorbitol, starch, pre-gelatinized starch, or mixtures thereof;

DISINTEGRANTS: agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, clays, other algins, other celluloses, gums, or mixtures thereof;

LUBRICANTS: calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil e.g., peanut oil, cottonseed oil, sunflower oil, sesame oil, olive oil, corn oil and soybean oil), zinc stearate, ethyl oleate, ethyl laurate, agar, syloid silica gel (AEROSIL 200, W.R. Grace Co., Baltimore, Md. USA), a coagulated aerosol of synthetic silica ([)eaussa Co., Plano, Tex. USA), a pyrogenic silicon dioxide (CAB-O-SIL, Cabot Co., Boston, Mass. USA), or mixtures thereof;

ANTI-CAKING AGENTS: calcium silicate, magnesiun silicate, silicon dioxide, colloidal silicon dioxide, talc, or mixtures thereof;

ANTIMICROBIAL AGENTS: benzalkonium chloride, benzethonium chloride, benzoic acid, benzyl alcohol, butyl paraben, cetylpyridinium chloride, cresol, chlorobutanol, dehydroacetic acid, ethylparaben, methylparaben, phenol, phenylethyl alcohol, phenylmercuric acetate, phenylmercuric nitrate, potassium sorbate, propylparaben, sodium benzoate, sodium dehydroacetate, sodium propionate, sorbic acid, thimersol, thymo, or mixtures thereof; and

COATING AGENTS: sodium carboxymethyl cellulose, cellulose acetate phthalate, ethylcellulose, gelatin, pharmaceutical glaze, hydroxypropyl cellulose, hydroxypropyl methylcellulose, hydroxypropyl methyl cellulose phthalate, methylcellulose, polyethylene glycol, polyvinyl acetate phthalate, shellac, sucrose, titanium dioxide, carnuba wax, microcrystalline wax, or mixtures thereof.

The dose range for adult humans is generally from 0.005 mg to 10 g/day orally. Tablets or other forms of presentation provided in discrete units may conveniently contain an amount of compound of the invention which is effective at such dosage or as a multiple of the same, for instance, units containing 5 mg to 500 mg, usually around 1Omg to 200mg. The precise amount of compound administered to a patient will be the responsibility of the attendant physician. However, the dose employed will depend on a number of factors, including the age and sex of the patient, the precise disorder being treated, and its severity.

Combination therapy can be achieved by administering two or more agents, each of which is formulated and administered separately, or by administering two or more agents in a single formulation. Other combinations are also encompassed by combination therapy. For example, two agents can be formulated together and administered in conjunction with a separate formulation containing a third agent. While the two or more agents in the combination therapy can be administered simultaneously, they need not be. For example, administration of a first agent (or combination of agents) can precede administration of a second agent (or combination of agents) by minutes, hours, days, or weeks. Thus, the two or more agents can be administered within minutes of each other or within 1, 2, 3, 6, 9, 12, 15, 18, or 24 hours of each other or within 1, 2, 3, 4, 5, 6, 7, 8, 9, 10, 12, 14 days of each other or within 2, 3, 4, 5, 6, 7, 8, 9, or 10 weeks of each other. In some cases even longer intervals are possible. While in many cases it is desirable that the two or more agents used in a combination therapy be present in within the patient's body at the same time, this need not be so. Combination therapy can also include two or more administrations of one or more of the agents used in the combination. For example, if agent X and agent Y are used in a combination, one could administer them sequentially in any combination one or more times, e.g., in the order X—Y—X, X—X—Y, Y—X—Y, Y—Y—X, X—X—Y—Y, etc.

In Vivo Assay of Hypolipidemic Agents using the Rat Cholesterol Absorption Model. This model is based on models described by Burnett et al (2002), Bioorg Med Chem Lett. Feb. 11, 2002;12(3):315-8 and J Lipid Res. October 1999;40(10):1747-57. Female Sprague-Dawley rats weighing 150-250g are separated into groups of 3-6 and fasted overnight. The animals (4-6/group) are dosed perorally with 300 μL test compounds in olive oil or suitable vehicle. Thirty minutes later, 3-5 microCuries ³H-cholesterol per rat are delivered perorally in 300 μL olive oil. After three hours, 200 μL serum is collected, vortexed with scintillation fluid, and measured for radioactivity in a scintillation counter. Percent inhibition is defined as 100*(1−Cₜₑₛₜ/C_{ctri}), where Cₜₑₛₜ and C_{ctri} refer to ³H levels in serum for the test compound and for the vehicle only control, respectively. Percent inhibition values are reported for a fixed dose. The ED₅₀ is the dose at which the half-maximal effect on serum ³H levels is observed for a given test compound.

In Vivo Assay of Hypolipidemic Agents using the Mouse Cholesterol Absorption Model. Female CD-1 mice weighing 20-30 g are separated into groups of 3-8 and fasted overnight. The animals (3-8/group) are dosed perorally with 200 μL test compound in olive oil or suitable vehicle. Thirty minutes later, 3-5 microCuries ³H-cholesterol per mouse are delivered perorally in 200 μL olive oil. After three hours, 100 μL serum is collected, vortexed with scintillation fluid, and measured for radioactivity in a scintillation counter. Percent inhibition and ED₅₀ are defined as in the Rat Cholesterol Absorption Model above.

In Vivo Assay of Hypolipidemic Agents Using the Hyperlipidemic Hamster: Hamsters are separated into groups of six and given a controlled cholesterol diet (Purina Chow #5001 containing 0.5% cholesterol) for seven days. Diet consumption is monitored to determine dietary cholesterol exposure in the face of test compounds. The animals are dosed with the test compound once daily beginning with the initiation of diet. Dosing is by oral gavage of 0.2 mL of corn oil alone (control group) or solution (or suspension) of test compound in corn oil. All animals moribund or in poor physical condition are euthanized. After seven days, the animals are anesthetized by intramuscular (IM) injection of ketamine and sacrificed by decapitation. Blood is collected into vacutainer tubes containing EDTA for plasma lipid analysis and the liver excised for tissue lipid analysis. Lipid analysis is conducted as per published procedures [Schnitzer-Polokoff, R., et al, Comp. Biochem. Physiol., 99A, 4, 665-670 (1991)] and data are reported as percent reduction of lipid versus control.

In Vivo Assay of Hypolipidemic Agents using the Hamster Acute Cholesterol Absorption Model. Male Syrian Hamsters weighing 120 g are separated into groups of 3-6 and fasted overnight. The animals (3-6/group) are dosed perorally with 200 μL test compound in olive oil or suitable vehicle. Thirty minutes later, 3-5 microCuries ³H-cholesterol per hamster are delivered perorally in 200 μL olive oil. After three hours, 100-200 μL serum is collected, vortexed with scintillation fluid, and measured for radioactivity in a scintillation counter. Percent inhibition and ED₅₀ are defined as in the Rat Cholesterol Absorption Model above.

The bioabsorption of the compounds herein described may be examined using the Caco-2 cell monolayer model of Hilger et al. [Pharm. Res. 7 902 (1990)].

To study the pharmacokinetics of compounds, bioavailability studies are carried out in rats. Compounds are prepared in suitable formulations: 5% ethanol in olive oil for oral administration and 2% DMSO: 20% cyclodextrins in H₂O for intravenous administration. Compounds are administered intravenously via tail vein injection and orally by gavage to independent groups of CD rats (200-250 g). Serum is collected at various time points and assayed for the presence of compounds using an LC/MS/MS detection method. Samples are diluted 15-fold in 30% acetonitrile in water, then injected (35 μL) into a 3.2 ml/min flow of 5% methanol in water onto a sample extraction cartridge (Waters Oasis HLB Direct Connect), washed for 30 seconds, then loaded onto a reverse phase HPLC column (Thermo Electron Betasil C18 Pioneer 50×2.1 mm, 5 um particle size). Samples are eluted from the reverse phase HPLC column with a gradient: (Mobile Phase A: 5 mM ammonium acetate in dH₂O, Mobile Phase B: 20% methanol in acetonitrile; 40% B ramping to 95% B over 4 minutes, and holding for 3 minutes, then returning to initial conditions to re-equilibrate the column for 1 min, all at a flow rate of 0.3 ml/min.). A Micromass Quattro Micro (Waters Corp.; Milford, Mass.) triple quadrupole mass spectrometer operated in MRM mode is used for detection. Concentrations are calculated based on standard concentration curves of compounds. MassLynx software (Waters, Corp.; Milford, Mass.) is used to calculate the absolute concentration of test compound in each serum sample. A concentration versus time plot is generated from the data in Microsoft Excel, Summit Software PK Solutions 2.0 or GraphPad Prism (GraphPad Software, Inc., San Diego, Calif.) to generate pharmacokinetic curves. An area under the curve (AUCₙ, n=length of experiment in minutes or hours) is calculated from the concentration vs. time data by the software using the trapezoid method for both the orally and intravenously dosed animals. Oral Bioavailability (F) over the length of the experiment is calculated using the equation:
F=(AUCₒᵣₐₗ*Dose_{i.v.})/(AUC_{i.v.}*Doseₒᵣₐₗ)

In general, the compounds of the present invention may be prepared by the methods illustrated in the general reaction schemes as, for example, described below, or by modifications thereof, using readily available starting materials, reagents and conventional synthesis procedures. In these reactions, it is also possible to make use of variants that are in themselves known, but are not mentioned here.

The starting materials, in the case of suitably substituted azetidinones, may be obtained by the methods described in WO 02/50027, WO 97/16424, WO 95/26334, WO 95/08532 and WO 93/02048, the disclosures of which are incorporated herein by reference.

Processes for obtaining the compounds of the invention are presented below. 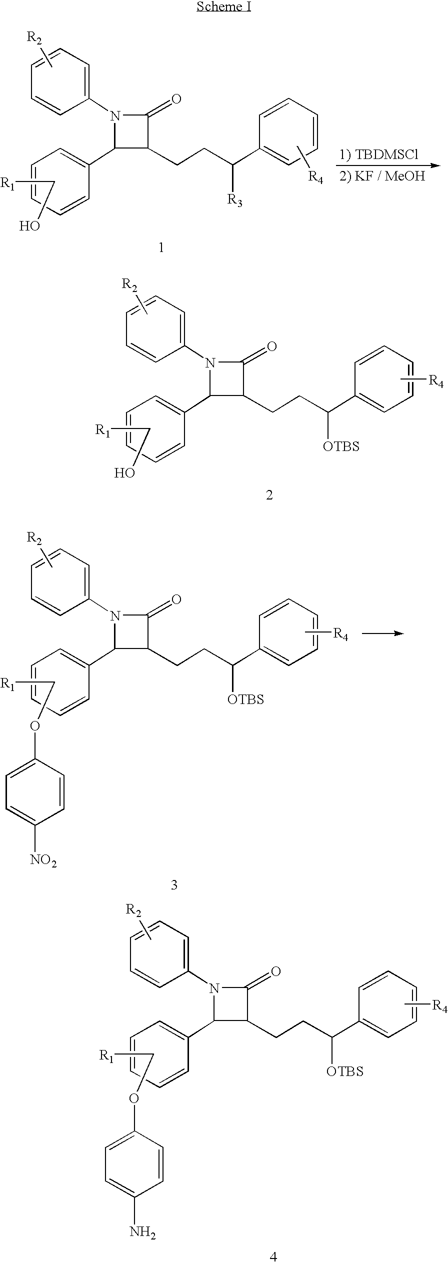 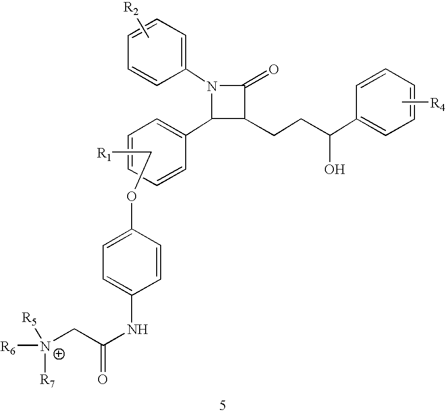
The method for the preparation of cholesterol absorption inhibitors illustrated in Scheme I begins with the double protection of the phenolic and benzylic hydroxyl moieties as tert-butyldimethylsilyl ethers (TBS). The bis-TBS ether is then subjected to treatment with methanolic potassium fluoride, which effects a selective deprotection of the phenol hydroxyl to afford compounds of the general formula 2. Treatment of the protected phenols (2) with 4-fluoronitrobenzene in the presence of base provided the nitrophenyl ether derivatives 3 which were converted to the corresponding anilines (4) upon catalytic hydrogenation. Acylation of aniline 4 with chloro or bromoacetic acid gave the corresponding alpha-halo acetyl derivatives, which were condensed with amines to afford the ammonium salts that were deprotected to the cholesterol absorption inhibitors 5. Alternatively, the anilines 4 could be condensed with alpha-ammonium salts of acetic acid and then deprotected to give the desired inhibitors 5. 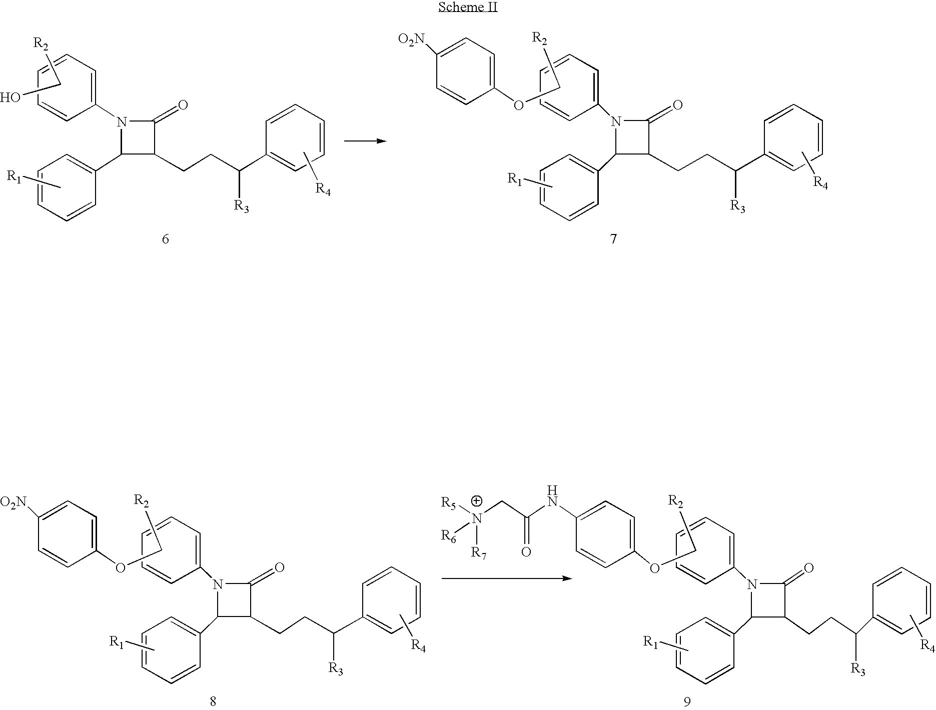
Scheme II illustrates the method that was used for the preparation of cholesterol absorption inhibitors that are isomeric with the derivatives described in Scheme I. The mono-protected phenol 6 was condensed with 4-fluoronitrobenzene to provide phenyl ethers 7. Catalytic hydrogenation of 7 afforded the corresponding anilines 8 which were converted into the cholesterol absorption inhibitors 9 as described in Scheme I. 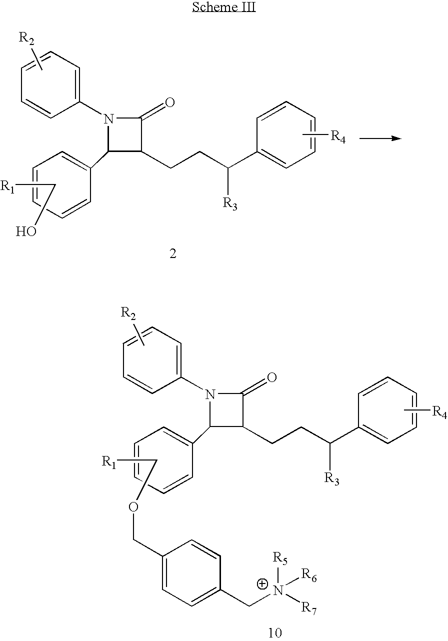
Scheme III illustrates the preparation of cholesterol absorption inhibitors of the type exemplified by quaternary ammonium salt 10. The synthesis begins with the unprotected phenols of the formula 1. Treatment of 1 with α,α′-dibromo-p-xylene followed by condensation with a tertiary amine afforded compounds of the formula 10. Deprotection of the benzylic TBS ether (if present) then affords the desired cholesterol absorption inhibitors 10 wherein R₃═OH. 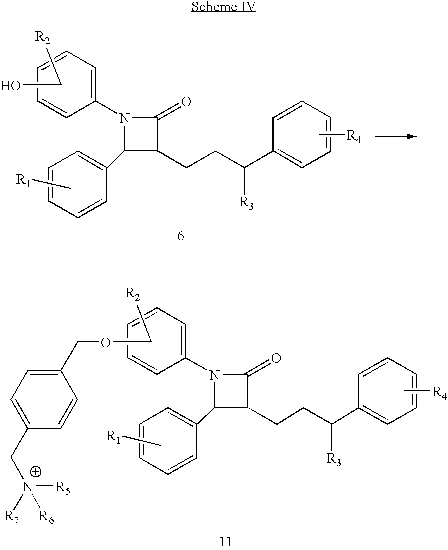
Scheme IV illustrates the preparation of cholesterol absorption inhibitors of the type exemplified by quaternary ammonium salt 11. The synthesis begins with phenols of the formula 6. Treatment of 6 with α,α′-dibromo-p-xylene followed by condensation with a tertiary amine afforded compounds of the formula 11. Deprotection of the benzylic TBS ether (if present) then affords the desired cholesterol absorption inhibitors 11 wherein R₃═OH. 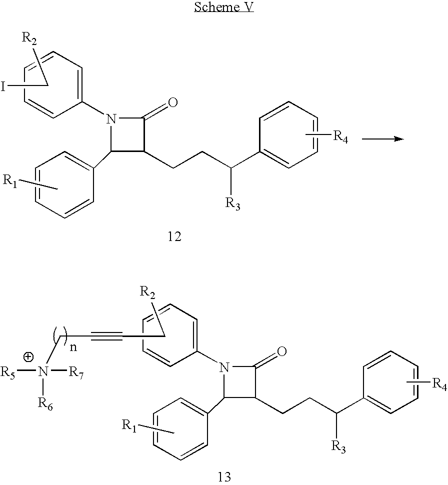
Scheme V illustrates the method that was employed for the preparation of cholesterol absorption inhibitors of the type exemplified by structure 13. The synthesis commences with condensation of an acetylenic derivative with an iodo substituted derivative 12 under Sonogoshira reaction conditions. The acetylene-substituted products were then converted into the corresponding ammonium salts (13) by quaternization of the appropriate bromide or sulfonate ester derivatives with an appropriate amine. Deprotection of the benzylic TBS ether (if present) then affords the desired cholesterol absorption inhibitors 13 wherein R₃═OH. 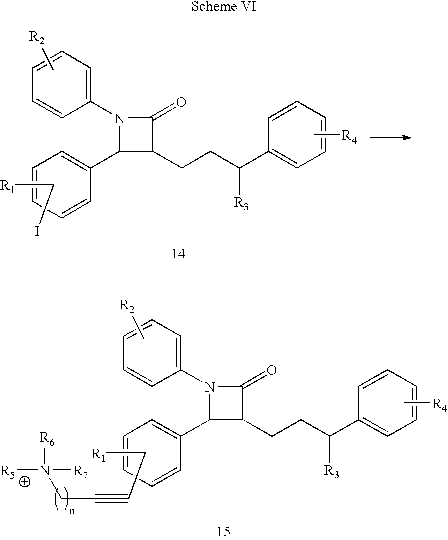
Scheme VI illustrates the method that was employed for the preparation of cholesterol absorption inhibitors of the type exemplified by structure 15. The synthesis commences with condensation of an acetylenic derivative with an iodo substituted derivative 14 under Sonogoshira reaction conditions. The acetylene-substituted products were then converted into the corresponding ammonium salts (15) by quaternization of the appropriate bromide or sulfonate ester derivatives with an appropriate amine. Deprotection of the benzylic TBS ether (if present) then affords the desired cholesterol absorption inhibitors 15 wherein R₃═OH. 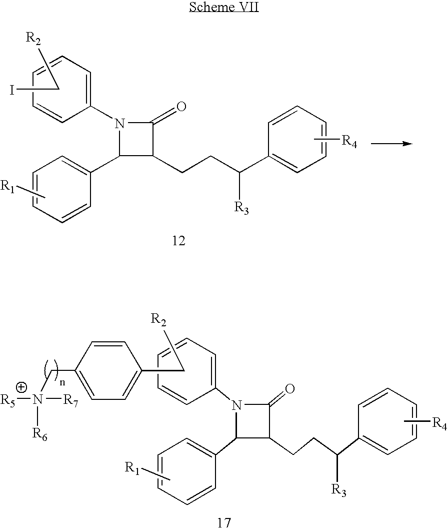
Scheme VII illustrates the method utilized for the preparation of cholesterol absorption inhibitors of the type exemplified by structure 17. The sequence commences by coupling the aromatic iodide 12 with an appropriately substituted aryl or alkyl boronic acid by Suzuki reaction conditions. Conversion of the coupling product to the desired ammonium salts 17 was then accomplished by preparation of the corresponding aryl bromide or sulfonate ester derivatives followed by quaternization with an appropriate amine. Deprotection of the benzylic TBS ether (if present) then affords the desired cholesterol absorption inhibitors 17 wherein R₃═OH. 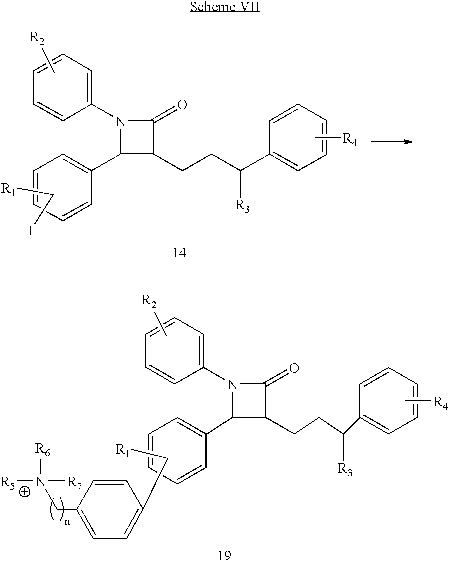
Scheme VIH illustrates the method utilized for the preparation of cholesterol absorption inhibitors of the type exemplified by structure 19. The sequence commences by coupling the aromatic iodide 14 with an appropriately substituted aryl or alkyl boronic acid by Suzuki reaction conditions. Conversion of the coupling product to the desired ammonium salts 19 was then accomplished by preparation of the corresponding aryl bromide or sulfonate ester derivatives followed by quaternization with an appropriate amine. Deprotection of the benzylic TBS ether (if present) then affords the desired cholesterol absorption inhibitors 19 wherein R₃═OH. 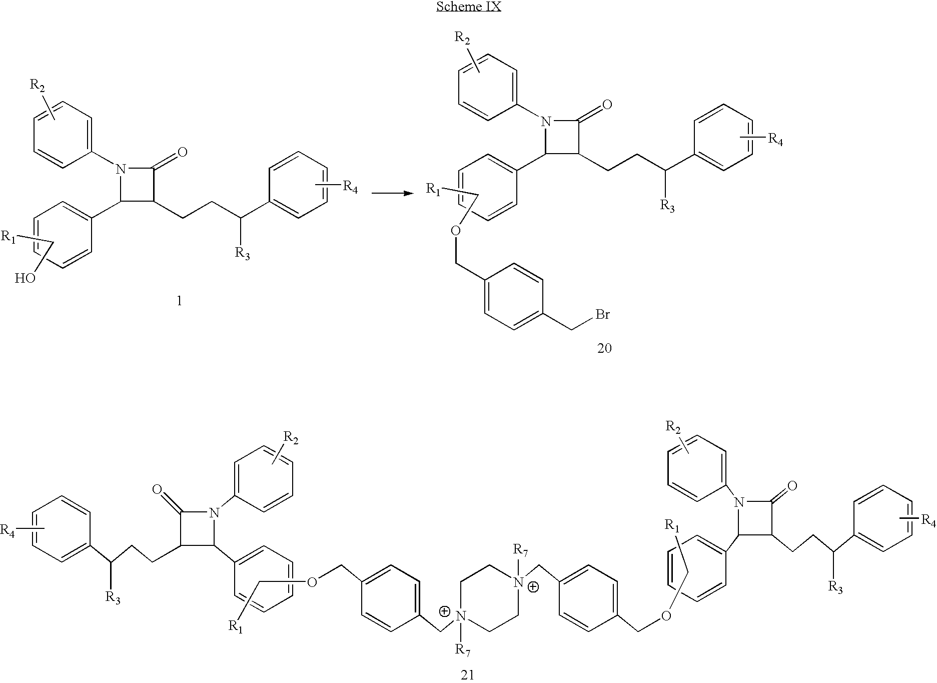
Illustrated in Scheme IX is the method that was employed to prepare compounds of the general formula 21. The sequence commences with compound 1 or if desired the protected form 2 (from Scheme I) by treatment with α,α′-dibromo-p-xylene to afford the mono-bromo derivative 20. Treatment of 20 with the quaternary ammonium salt 10 where in R₇, R8 and R₉ are derived from an N,N′ disubstituted piperazine derivative gave the desired bis-ammonium salts 21. Deprotection of the benzylic TBS ether (if present) then affords the desired cholesterol absorption inhibitors 21 wherein R₃═OH. 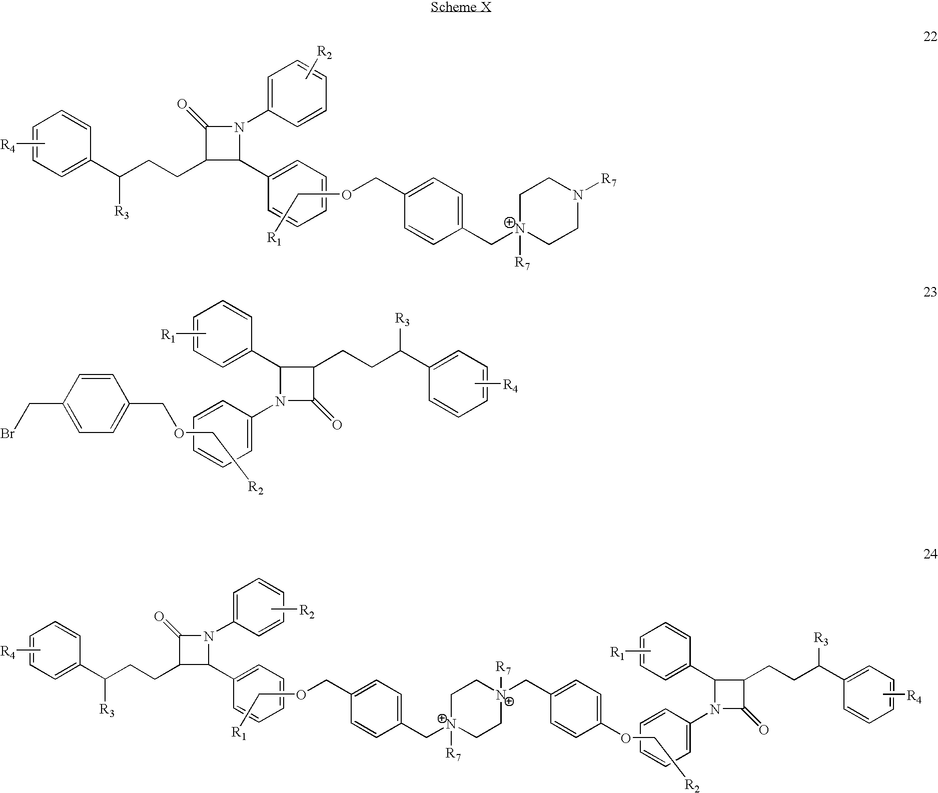
Illustrated in Scheme X is the general method that was used for the preparation of bis-ammonium salts of the general formula 24. Treatment of a-bromoxylene derivatives (20) with an N,N′ disubstituted piperazine derivative affords salts of the type 22. Condensation of the salts 22 with a-bromoxylene derivatives of the type 23 (prepared from phenols 6 and α,α′-dibromo-p-xylene) provides the desired bis-salts 24. Deprotection of the benzylic TBS ether (if present) then affords the desired cholesterol absorption inhibitors 24 wherein R₃OH. 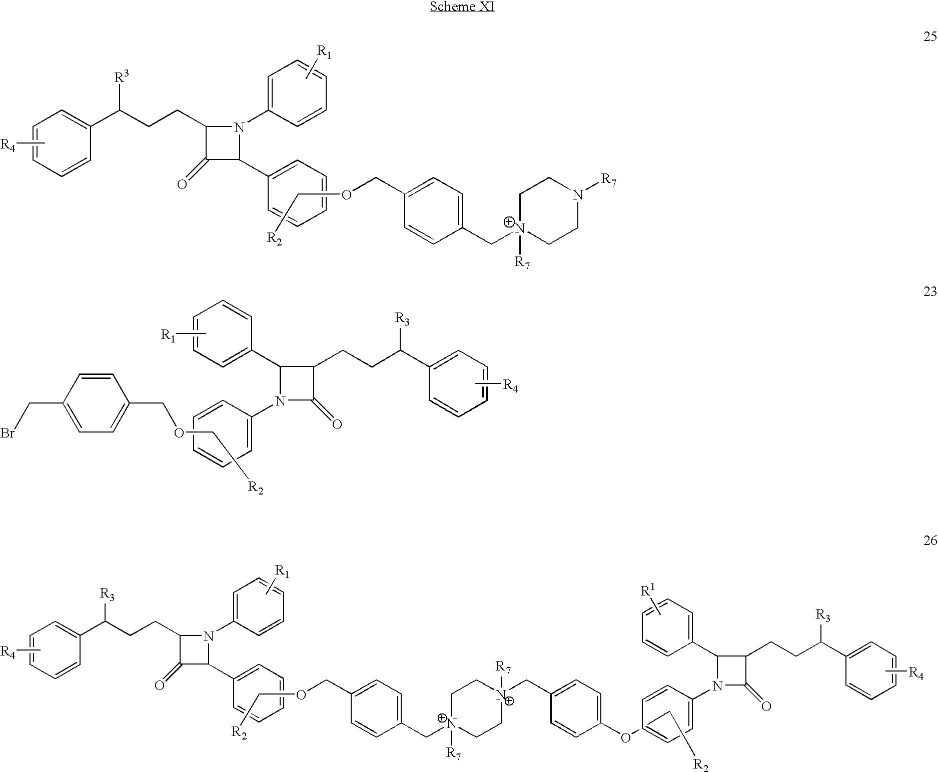
Illustrated in Scheme XI is the general method that was used for the preparation of bis-ammonium salts of the general formula 26. Treatment of α-bromoxylene derivatives (23) with an N,N′ disubstituted piperazine derivative affords salts of the type 25. Condensation of the salts 25 with Ct-bromoxylene derivatives 23 provides the desired bis-salts 26. Deprotection of the benzylic TBS ether (if present) then affords the desired cholesterol absorption inhibitors 26 wherein R₃═OH. 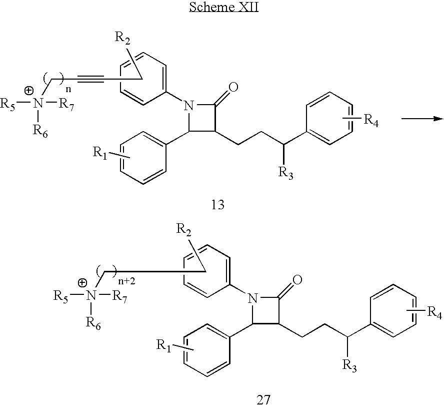
Illustrated in Scheme XII is the general method for the preparation of ammonium salts of the general formula 27. The method for the preparation of these analogues is catalytic hydrogenation of the corresponding acetyleneic salts 13. Deprotection of the benzylic TBS ether (if present) then affords the desired cholesterol absorption inhibitors 27 wherein R₃═OH. 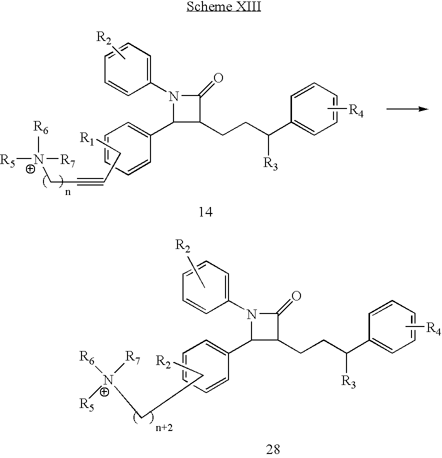
Illustrated in Scheme XII is the general method for the preparation of ammonium salts of the general formula 28. The method for the preparation of these analogues is catalytic hydrogenation of the corresponding acetyleneic salts 14. Deprotection of the benzylic TBS ether (if present) then affords the desired cholesterol absorption inhibitors 28 wherein R₃═OH. 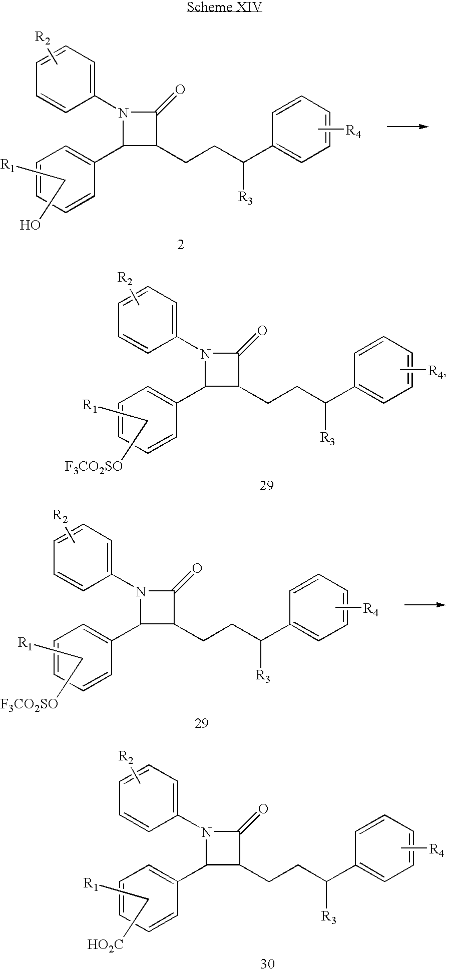
Illustrated in Scheme XIV is the general method that was employed for the preparation of carboxylic acids of the type exemplified by compound 30. The sequence commences with the conversion of phenols of the general formula 2 to their corresponding trifluoromethane sulfonate esters 29 upon treatment with N-phenyltrifluoromethanesulfonimide in the presence of triethylamine. The triflate 29 is then converted into a carboxylic acid by dissolving in dimethyl sulfoxide and treatment with carbon monoxide in the presence of palladium II acetate and 1,1-bis-(diphenylphosphino)ferrocene (dppf). Deprotection of the benzylic TBS ether then affords the desired cholesterol absorption inhibitors 30 wherein R₃═OH. The carboxylic acids of the type 30 are also useful intermediates for the preparation of cholesterol absorption inhibitors. 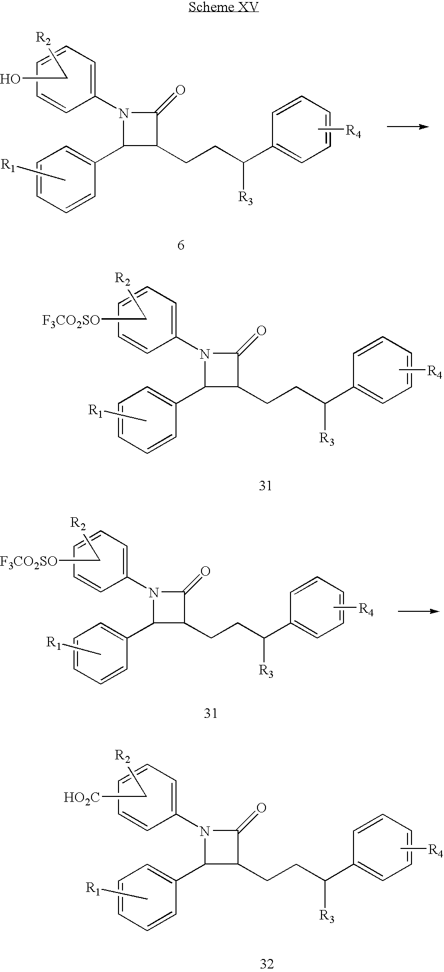
Illustrated in Scheme XV is the general method that was employed for the preparation of carboxylic acids of the type exemplified by compound 32. The sequence commences with the conversion of phenols of the general formula 2 to their corresponding trifluoromethane sulfonate esters 31 upon treatment with N-phenyltrifluoromethanesulfonimide in the presence of triethylamine. The triflate 31 is then converted into a carboxylic acid by dissolving in dimethyl sulfoxide and treatment with carbon monoxide in the presence of palladium II acetate and 1,1-bis-(diphenylphosphino)ferrocene (dppf). Deprotection of the benzylic TBS ether then affords the desired cholesterol absorption inhibitors 32 wherein R₃═OH. The carboxylic acids of the type 32 are also useful intermediates for the preparation of cholesterol absorption inhibitors.

The trifluoromethane sulfonate esters 29 and 31 are also useful for the preparation of acetylene substituted cholesterol absorption inhibitors 13 and 15 by the Sonogoshira reaction. In addition, the Suzuki coupling method may employ 29 and 31 as coupling partners for the preparation of cholesterol absorption inhibitors 17 and 19. 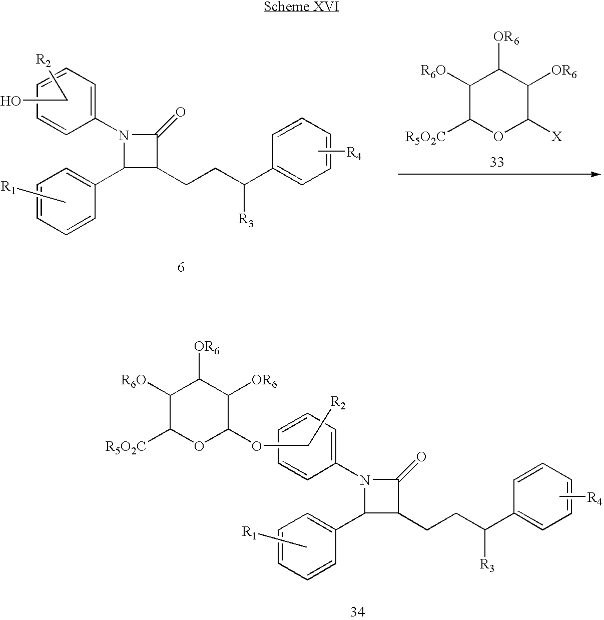
Illustrated in Scheme XVI is the general method for the preparation of cholesterol absorption inhibitors of the general formula 34. The synthesis commences with the coupling of a donor phenol 6 with an activated sugar derivative 33 to effect coupling to afford the protected compounds 34. The activating group may be, for example, —OCNHCCl₃. Subsequent deprotection, if desired, affords the cholesterol absorption compounds 34. Reactions for adding and deprotecting sugars are described in U.S. Pat. No. 5,756,470, which is incorporated herein by reference. 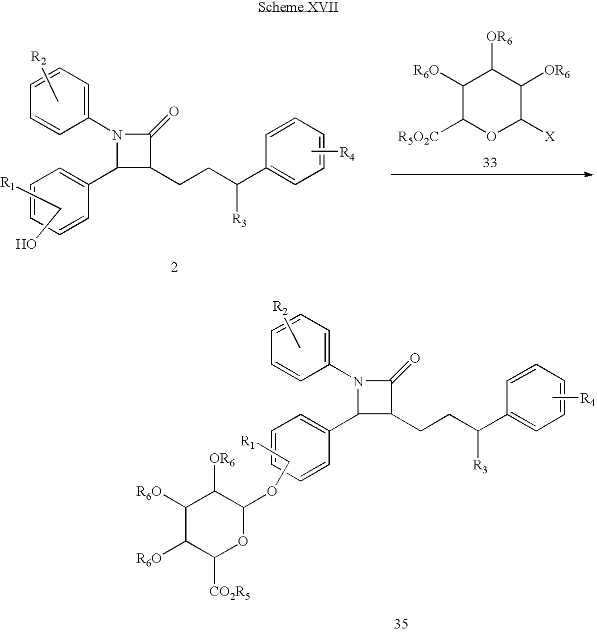
Illustrated in Scheme XVII is the general method for the preparation of cholesterol absorption inhibitors of the general formula 35. The synthesis commences with the coupling of a donor phenol 2 with an activated sugar derivative 33 to effect coupling to afford the protected compounds 35. Subsequent deprotection, if desired, affords the cholesterol absorption compounds 35. 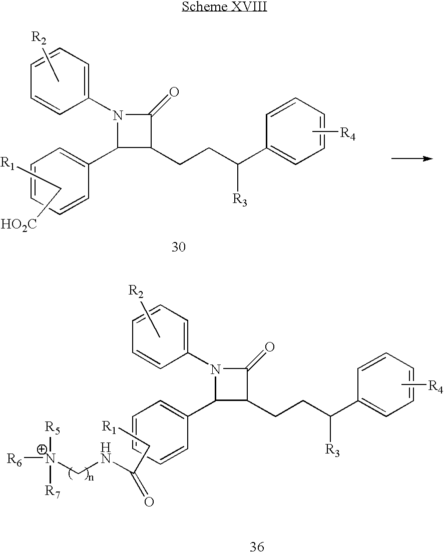
Illustrated in Scheme XVIII is the general synthetic method for the preparation of cholesterol absorption inhibitors of the formula 36. The method involves coupling of the carboxylic acids 30 with an amino substituted ammonium salt under amide forming conditions to afford the desired salts 36. Alternatively, the acids 30 can be coupled with a tertiary amine containing primary amine followed by quaternization with an alkyl halide (R7) to afford the salts 36. 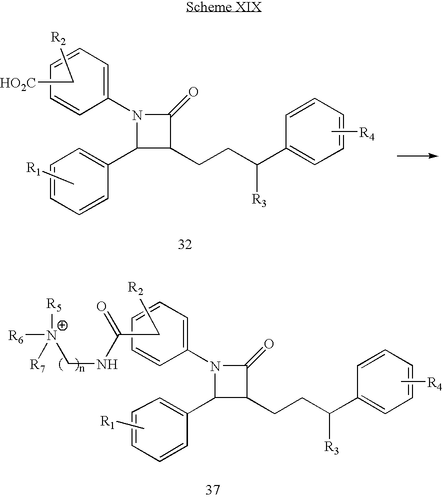
Illustrated in Scheme XIX is the general synthetic method for the preparation of cholesterol absorption inhibitors of the formula 37. The method involves coupling of the carboxylic acids 32 with an amino substituted ammonium salt under amide forming conditions to afford the desired salts 37. Alternatively, the acids 32 can be coupled with a tertiary amine containing primary amine followed by quaternization with an alkyl halide (R7) to afford the salts 37. 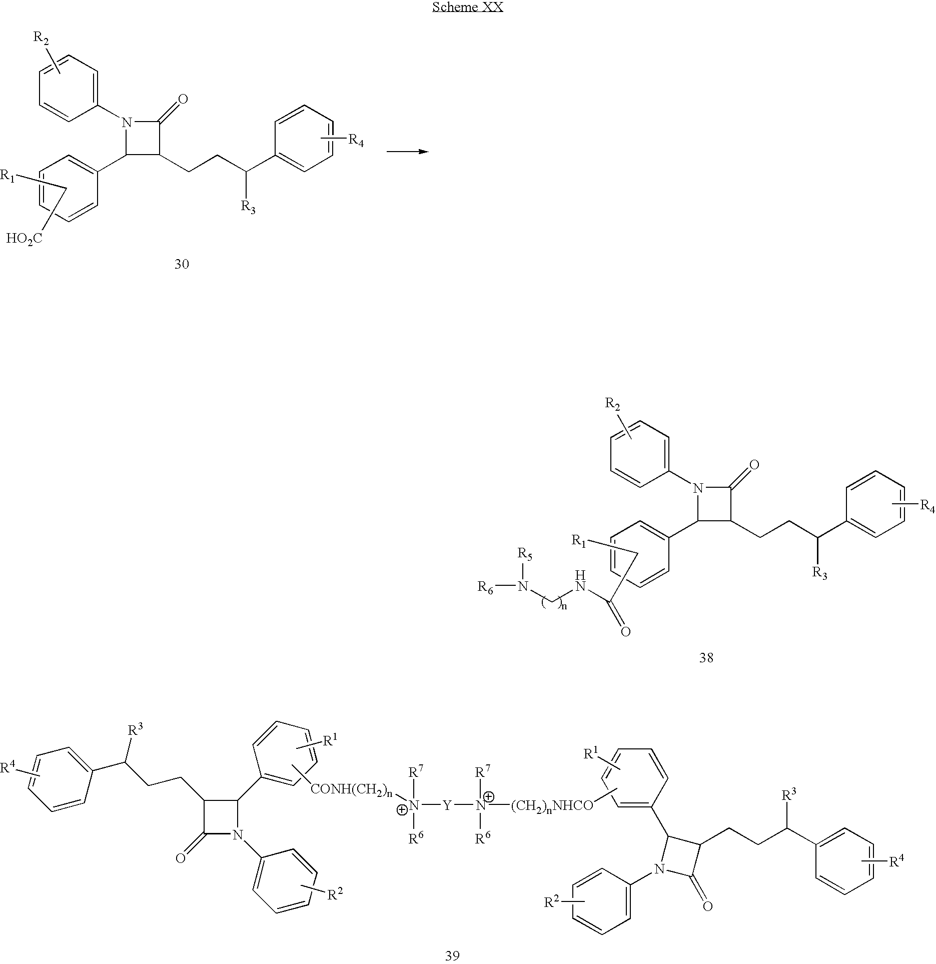
Illustrated in Scheme XX is the general synthetic method for the preparation of cholesterol absorption inhibitors of the formula 39. The method involves coupling of the carboxylic acids 30 with a tertiary amine containing primary amine followed by quaternization with a bis-alkyl halide (represented by the X in structure 39) to afford the bis-salts 39. Examples of bis-alkyl halides would be useful for the preparation of the bis-salts 39 would be compounds such as 1,4-dibromobutane, 1,3-dibromopropane, α,α′-dibromo-para-xylene, α,α′-dibromo-meta-xylene and the like. 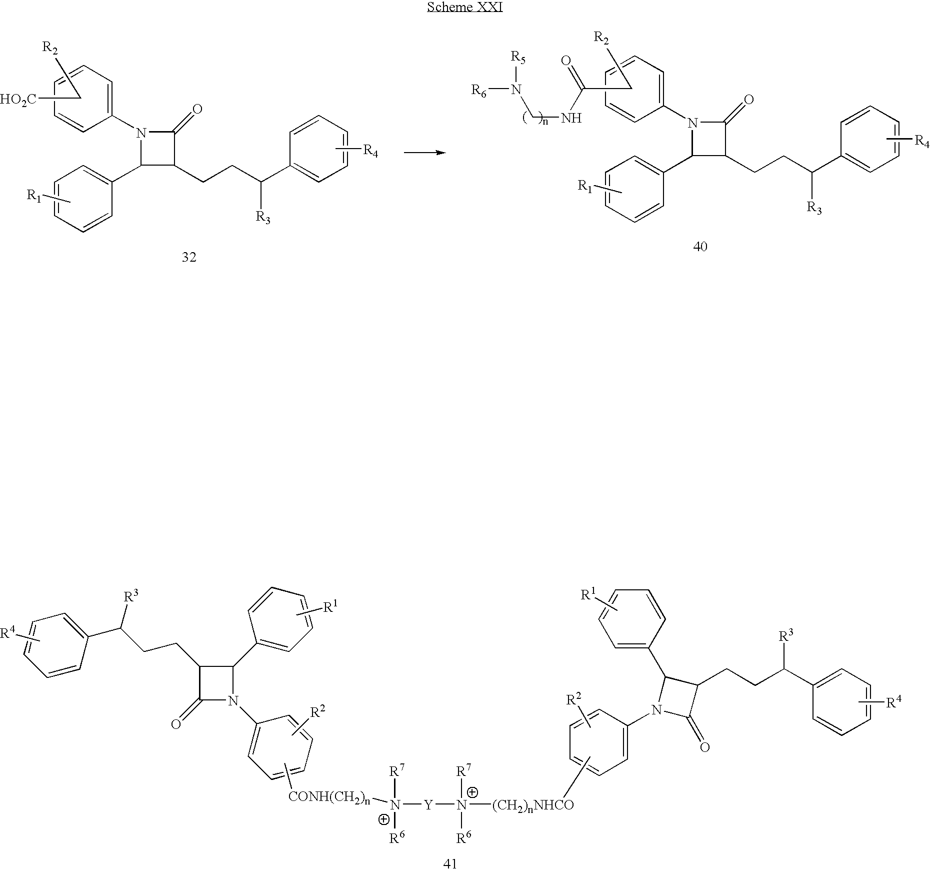
Illustrated in Scheme XX is the general synthetic method for the preparation of cholesterol absorption inhibitors of the formula 41. The method involves coupling of the carboxylic acids 32 with a tertiary amine containing primary amine followed by quaternization with a bis-alkyl halide (represented by the X in structure 41) to afford the bis-salts 41. Examples of bis-alkyl halides would be useful for the preparation of the bis-salts 41 would be compounds such as 1,4-dibromobutane, 1,3-dibromopropane, α,α′-dibromo-para-xylene, α,α′-dibromo-meta-xylene and the like. 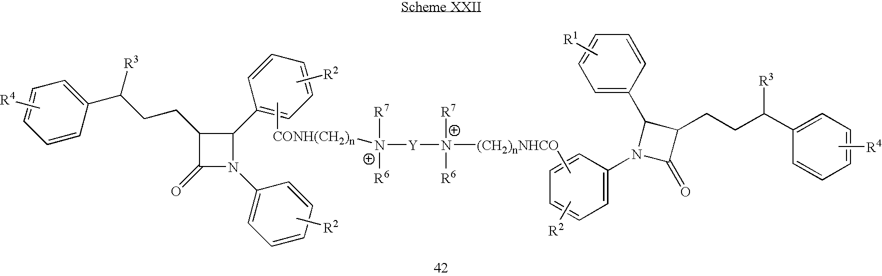
The compound illustrated in Scheme XXII as a bis-quaternary ammonium salt cholesterol absorption inhibitor, 42. The preparation of 42 can be effected by treatment of a mixture of the tertiary amine containing derivatives 38 and 40 with a bis-alkyl halide. Examples of bis-alkyl halides would be useful for the preparation of the bis-salts 42 would be compounds such as 1,4-dibromobutane, 1,3-dibromopropane, α,α′-dibromo-para-xylene, α,α′-dibromo-meta-xylene and the like.

### Preparation of 1-{4-[(4-{(2S,3R)-1-(4-fluorophenyl)-3-[(3S)-3-hydroxypropyl]-4-oxoazetidin-2-yl}phenoxy)methyl]benzyl}-1-azoniabicyclo[2.2.2]octane chloride

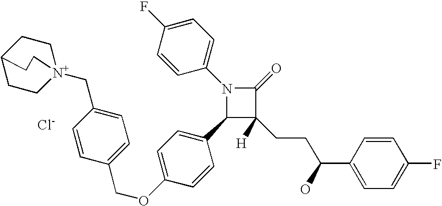

### Step 1. Preparation of (3R,4S)-4-[4-(4-bromomethyl benzyloxy)phenyl]-1-(4-fluorophenyl)-3-[(3S)-3-(4-fluorophenyl)-3-hydroxypropyl]azetidin-2-one

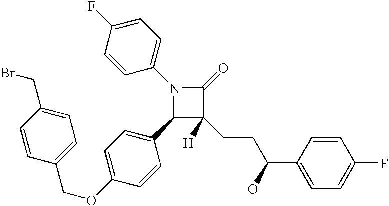

Cesium carbonate (344.7 mg, 1.06 mmol) was lightly flame-dried in a flame-dried flask. When cooled, N,N-dimethylformamide (DMF) (5.0 mL) was added via syringe followed by α,α′-dibromo-p-xylene (826.2 mg, 3.13 mmol) and finally (3R,4S)-1-(4-fluorophenyl)-3-[(3 S)-3-(4-fluorophenyl)-3-hydroxypropyl]-4-(4-hydroxyphenyl)azetidin-2-one (254.1 mg, 0.621 mmol) both as solids. The reaction was stirred for 3 h at room temperature, diluted with ethyl acetate (20 mL), filtered through a pad of Celiteg and washed with ethyl acetate (100 mL). The solution was transferred to a separatory funnel, washed with water (3×100 mL) and brine (50 mL), dried over sodium sulfate, filtered, concentrated and purified by chromatography (35 g silica gel, 10% to 90% ethyl acetate-hexane) to afford (3R,4S)-4-[4-(4-bromomethyl benzyloxy)phenyl]-1-(4-fluorophenyl)-3-[(3S)-3-(4-fluorophenyl)-3-hydroxypropyl]azetidin-2-one (265.3 mg, 72% yield) as a clear film.

### Step 2. Preparation 1-{4-[(4-{(2S,3R)-1-(4-fluorophenyl)-3-fluorophenyl)-3-hydroxypropyl]-4-oxoazetidin-2-yl}phenoxy)methyl]benzyl}-1-azoniabicyclo[2.2.2]octane chloride

The product from Step 1 (138.6 mg, 0.234 mmol) was dissolved in dry acetonitrile (1.0 mL). Quinuclidine (26.0 mg, 0.234 mmol) in 1.0 mL of dry acetonitrile was added to the bromide mixture and the reaction was stirred at room temperature for 5 h. The solution was concentrated, purified by reverse-phase HPLC (21 mm column, 35% to 65% acetonitrile-0.1% trifluoroacetic acid in water), passed through Dowex® 21K Cl (chloride) anion exchange resin in methanol and concentrated to afford 1-{4-[(4-{(2S,3R)-1-(4-fluorophenyl)-3-[(3S)-3-(4-fluorophenyl)-3-hydroxypropyl]-4-oxoazetidin-2-yl}phenoxy)methyl]benzyl}-1-azoniabicyclo[2.2.2]octane chloride (137.3 mg, 92% yield) as a glassy solid.

### Preparation of 1-{4-[(4-{(2S,3R)-1-(4-fluorophenyl)-3-[(3S)-3-(4-fluorophenyl)-3-hydroxypropyl]-4-oxoazetidin-2-yl}phenoxy)methyl]benzyl}-4-aza-1-azoniabicyclo[2.2.2]octane bromide

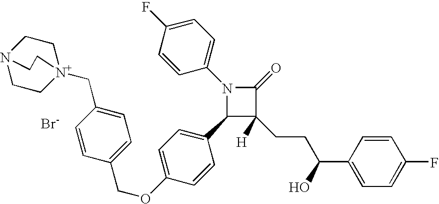 (3R,4S)-4-[4-(4-bromomethyl benzyloxy)phenyl]-1-(4-fluorophenyl)-3-[(3S)-3-(4-fluorophenyl)-3-hydroxypropyl]azetidin-2-one (55.9 mg, 0.094 mmol) was dissolved in dry acetonitrile (2.0 mL). A solution of 1,4-diazabicyclo[2.2.2]octane (9.5 mg, 0.085 mmol) in 0.5 mL of dry acetonitrile was added to the bromide mixture, the reaction was stirred at room temperature for 3 h and then concentrated. The residue was partitioned between water (30 mL) and 1:1 ethyl acetate-hexane (30 mL), shaken to form an emulsion and transferred to two 50-mL Falcon® tubes. The samples were spun at 3000 rpm for 25 min and the aqueous layers are removed carefully via pipette, combined, concentrated at 35° C. and azeotropically dried with methanol (20 mL) to afford 1-{4-[(4-{(2S,3R)-1-(4-fluorophenyl)-3-[(3 S)-3-(4-fluorophenyl)-3-hydroxypropyl]-4-oxoazetidin-2-yl}phenoxy)methyl]benzyl}-4-aza-1-azoniabicyclo[2.2.2]octane bromide (54.8 mg, 92% yield) as a clear film.

### Preparation of 1,4-bis{4-[(4-{(2S,3R)-1-(4-fluorophenyl)-3-[(3S)-3-(4-fluorophenyl)-3-hydroxypropyl]-4-oxoazetidin-2-yl}phenoxy)methyl]benzyl}-1,4-diazoniabicyclo[2.2.2]octane dibromide

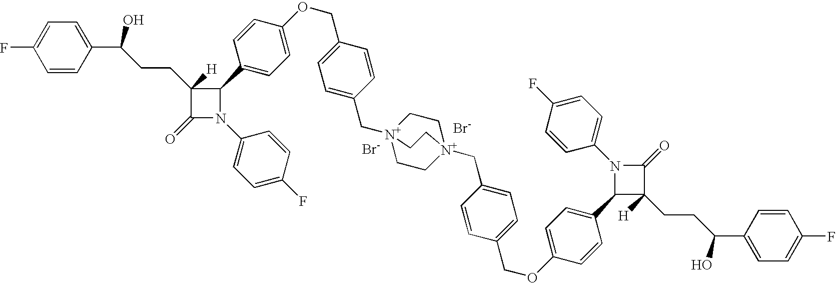 1-{4-[(4-{(2S,3R)-1-(4-fluorophenyl)-3-[(3S)-3-(4-fluorophenyl)-3-hydroxypropyl]-4-oxoazetidin-2-yl}phenoxy)methyl]benzyl}-4-aza-1-azoniabicyclo[2.2.2]octane bromide (79.2 mg, 0.112 mmol) was dissolved in dry acetonitrile (1.0 mL). A solution of (3R,4S)-4-[4-(4-bromomethyl benzyloxy)phenyl]-1-(4-fluorophenyl)-3-[(3 S)-3-(4-fluorophenyl)-3-hydroxypropyl]azetidin-2-one (74.2 mg, 0.125 mmol) in 2×3 mL of dry acetonitrile was added to the amine solution, the reaction was stirred at 50° C. for 3 h and then concentrated. The supernatant was decanted off and the remaining residue at the bottom of the flask was triturated with water (20 mL) and ethyl acetate (20 mL) and then azeotropically dried with methanol (20 mL) to afford 1,4-bis{4-[(4-{(2S,3R)-1-(4-fluorophenyl)-3-[(3 S)-3-(4-fluorophenyl)-3-hydroxypropyl]-4-oxoazetidin-2-yl}phenoxy)methyl]benzyl}-1,4-diazoniabicyclo[2.2.2]octane dibromide (131.9 mg, 91% yield) as a clear glassy solid.

1. A compound of formula 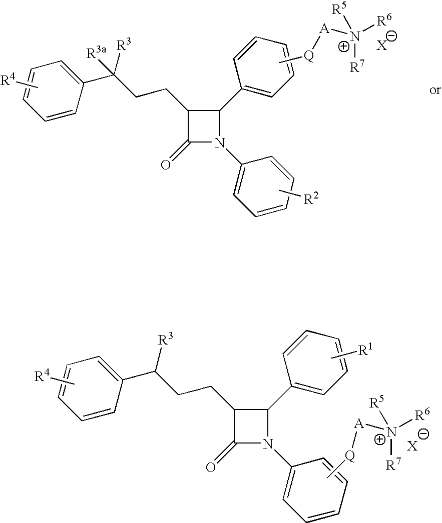
wherein
R¹ and R² are chosen from H, halogen, —OH, loweralkyl, —O-loweralkyl, —CN, —S-loweralkyl, amino, acyl, lower aminoalkyl, alkylsulfonyl, arylsulfonyl, a sugar, a glucuronide and a sugar carbamate;
R³ is chosen from H, —OH, fluoro and —O-loweralkyl;
R³a is chosen from H and fluoro, or R^{3a} and R³ together are ═O;
R⁴ is chosen from H, halogen, —OH, loweralkyl, —O-loweralkyl, —CN, —S-loweralkyl, amino, acyl and lower aminoalkyl, alkylsulfonyl, arylsulfonyl;
Q is chosen from a direct bond, —O—, —S—, —NH—, —CH₂O—, —CH₂NH—, —C(═O)—, —CONH—, —NHCO—, —O(C═O)—, —(C═O)O—, —NHCONH—, —OCONH— and —NHCOO—;
A is chosen from C₂ to C₂₀ hydrocarbon, substituted alkyl of 2 to 20 carbons, substituted aryl, substituted arylalkyl, and oxaalkyl of four to fifty carbons; and, when Q is a direct bond, C(═O) or —O(C═O)—, A may additionally be methylene;
R⁵ forms a five- to seven-membered ring with A or R⁶;
R⁶ is alkyl, forms a double bond with A or forms a five- to seven-membered ring with R⁵;
R⁷ is alkyl or together with R⁵ or R⁶ forms a second five- to seven-membered ring; and
when Q is not —O— or —CH₂NH—, R⁵,may additionally be alkyl or aryl; and
X is an anion.

2. A compound chosen from three isomers of formulae: 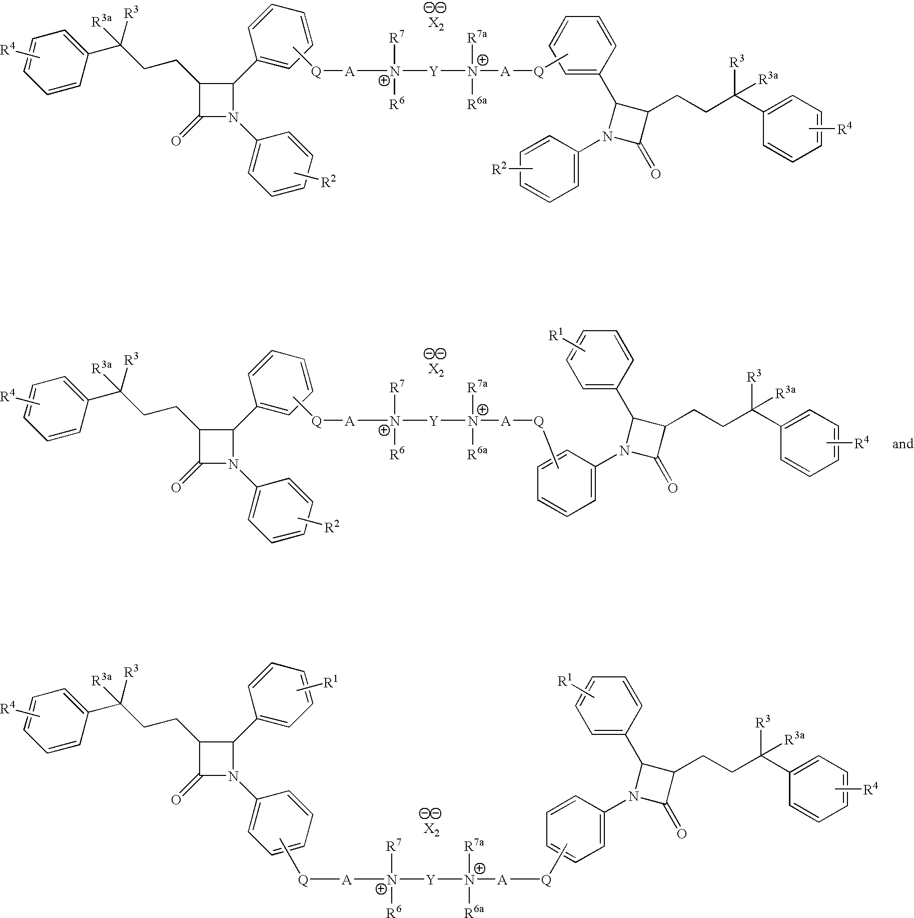
wherein
R¹ and R² are chosen from H, halogen, —OH, loweralkyl, —O-loweralkyl, —CN, —S-loweralkyl, amino, acyl, lower aminoalkyl, alkylsulfonyl, arylsulfonyl, a sugar, a glucuronide and a sugar carbamate;
R³ is chosen from H, —OH, fluoro and —O-loweralkyl;
R³a is chosen from H and fluoro, or R^{3a} and R³ together are ═O;
R⁴ is chosen from H, halogen, —OH, loweralkyl, —O-loweralkyl, —CN, —S-loweralkyl, amino, acyl and lower aminoalkyl, alkylsulfonyl, arylsulfonyl;
Q is chosen from a direct bond, —O—, —S—, —NH—, —CH₂O—, —CH₂NH—, —C(═O)—, —CONH—, —NHCO—, —O(C═O)—, —(C═O)O—, —NHCONH—, —OCONH— and —NHCOO—;
A is chosen from C₂ to C₂₀ hydrocarbon, substituted alkyl of 2 to 20 carbons, substituted aryl, substituted arylalkyl and oxaalkyl of four to fifty carbons; and, when Q is a direct bond, —C(═O) or —O(C═O)—, A may additionally be methylene;
Y is chosen from C₂ to C₂₀ hydrocarbon, substituted alkyl of 2 to 20 carbons, substituted arylalkyl and oxaalkyl of four to fifty carbons;
R⁶ and R⁶1 are alkyl or together with Y form a first five- to seven-membered ring;
R⁷ and R⁷a are alkyl or together form a second five- to seven-membered ring; and
X₂ is either a dianion or two monoanions.

3. A compound according to claim 2 chosen from three isomers of formulae:

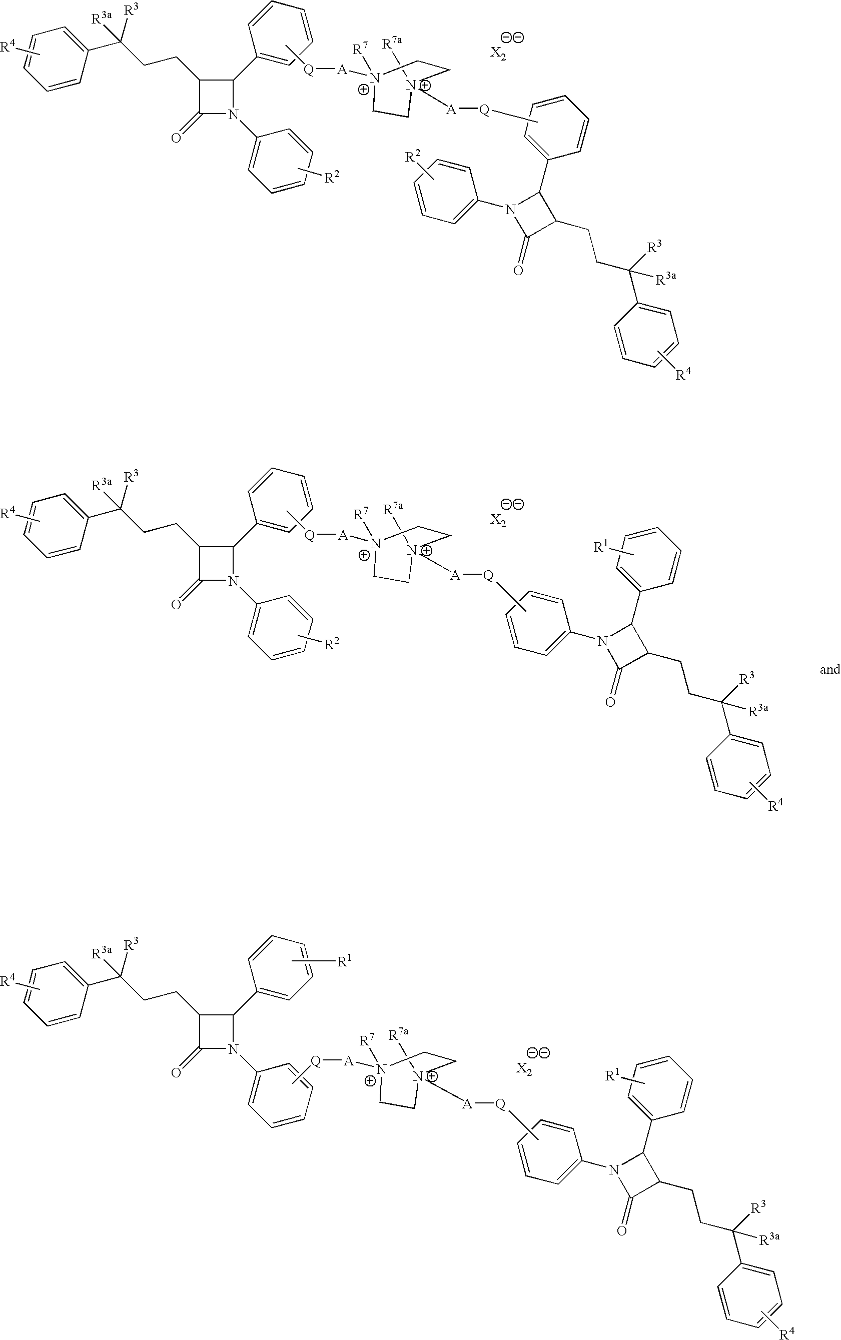

4. A compound according to any of claims 1, 2 or 3 wherein R⁷ forms a second six-membered ring.

5. A compound according to any of claims 1 to 4 wherein -Q-A- is chosen from (C₂ to C₂₀ hydrocarbon), —O—(C₂ to C₂₀ hydrocarbon), —NH(C₂ to C₂₀ hydrocarbon), —NHCO(C₂ to C₂₀ hydrocarbon) and oxaalkyl of four to fifty carbons.

6. A compound according to any of claims 1 to 5 wherein
R¹ and R² are chosen from H, halogen, —OH, and methoxy;
R³ is —OH; and
R⁴ is fluoro.

7. A compound according to any of claims 1 to 5 wherein
R¹ and R² are chosen from a sugar, a glucuronide and a sugar carbamate;
R³ is —OH; and
R⁴ is fluoro.

8. A compound according to any of claims 1 or 4 to 7 wherein R⁵,R⁶ and R⁷ taken together form a diazabicyclooctane quat:

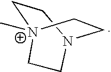

9. A compound according to any of claims 1 or 4 to 7 wherein R⁵,R⁶ and R⁷ talcen together form a quinuclidinium quat:

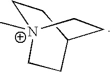

10. A compound according to any of claims 2 to 7 wherein R⁷ and R⁷a taken together form a diazabicyclooctane bisquat:

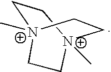

11. A compound according to claim 8 of formula:

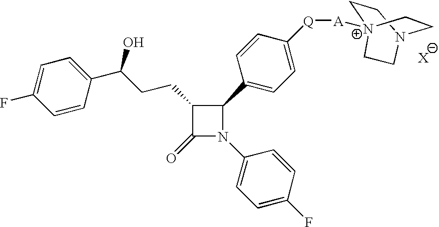

12. A compound according to claim 10 of formula:

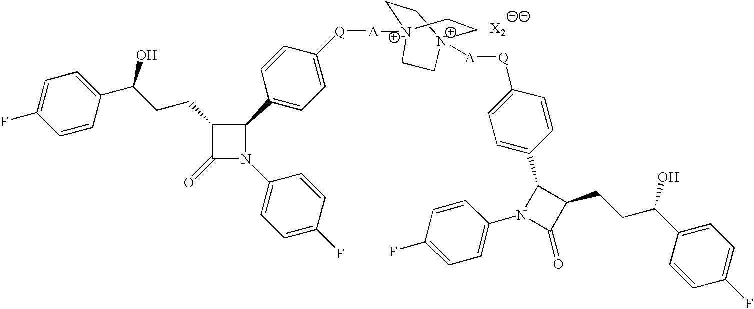

13. A compound according to any of claims 2 to 7 wherein R⁶, R^{6a}, R⁷ and R^{7a} are all and Y is chosen from C₂ to C₁₀ alkylene and xylylene.

14. A compound according to any of claims 1 to 13 wherein -Q-A- is

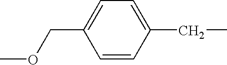

15. A compound according to any of claims 1, 6 or 7 wherein R⁵ forms a six-membered ring with A;
R⁶ forms a double bond with A; and
R⁷ is alkyl:

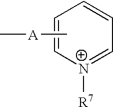

16. 1-{4-[(4-{(2S,3R)-I-(4-fluorophenyl)-3-[(3S)-3-(4-fluorophenyl)-3-hydroxypropyl]-4-oxoazetidin-2-yl}phenoxy)methyl]benzyl}-1-azoniabicyclo[2.2.2]octane chloride

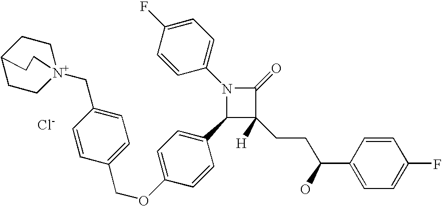

17. 1-{4-[(4-{(2S,3R)-1-(4-fluorophenyl)-3-[(3S)-3-(4-fluorophenyl)-3-hydroxypropyl]-4-oxoazetidin-2-yl}phenoxy)methyl]benzyl}-4-aza-1-azoniabicyclo[2.2.2]octane

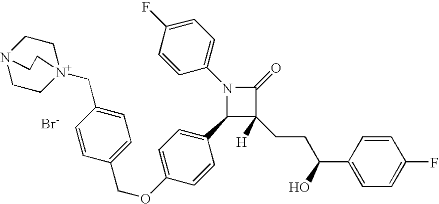

18. 1,4-bis{4-[(4-{(2S,3R)-1-(4-fluorophenyl)-3-[(3S)-3-(4-fluorophenyl)-3-hydroxypropyl]-4-oxoazetidin-2-yl}phenoxy)methyl]benzyl}-1,4-diazoniabicyclo[2.2.2]octane

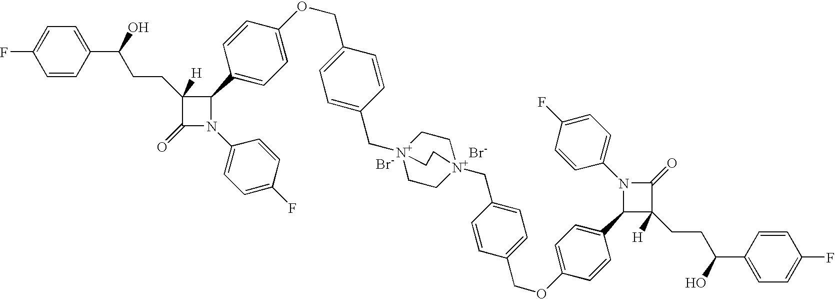

19. A compound according to any of claims 1 to 18 wherein X or X₂ is a pharmaceutically acceptable anion.

20. A compound according to claim 19 wherein X is an anion chosen from the group consisting of hydroxide, acetate, benzenesulfonate (besylate), benzoate, bicarbonate, bisulfate, carbonate, camphorsulfonate, citrate, ethanesulfonate, fumarate, gluconate, glutamate, bromide, chloride, isethionate, lactate maleate, malate, mandelate, methanesulfonate, mucate, nitrate, pamoate, pantothenate, phosphate, succinate, sulfate, tartrate and p-toluenesulfonate.

21. A compound according to any of claims 2 to 7, 10, 12, 13 or 19 wherein X₂ is a dianion chosen from the group consisting of carbonate, citrate, fumarate, lactate, maleate, malate, phosphate, succinate, sulfate and tartrate.

22. A pharmaceutical formulation comprising a compound according to any of claims 19 to 21 and a pharmaceutically acceptable carrier.

23. A pharmaceutical formulation according to claim 22 additionally comprising an inhibitor of cholesterol biosynthesis.

24. A method for treating a disorder of lipid metabolism comprising administering a to a mammal a therapeutically effective amount of a compound according to any of claims 19 to 21.

25. A method according to claim 24, wherein said disorder of lipid metabolism is hyperlipidemia.

26. A method according to claim 24, wherein said disorder of lipid metabolism is arteriosclerosis.

27. A method for inhibiting the absorption of cholesterol from the intestine of a mammal, which comprises administering an effective cholesterol-absorption-inhibiting amount of a compound according to any of claims 19 to 21 to the mammal.

28. A method for reducing the blood plasma or serum concentrations of LDL cholesterol in a mammal, which comprises administering an effective cholesterol reducing amount of a compound according to any of claims 19 to 21 to the mammal.

29. A method for reducing the concentrations of cholesterol and cholesterol ester in the blood plasma or serum of a mammal, which comprises administering and effective cholesterol and cholesterol ester reducing amount of a compound according to any of claims 19 to 21 to the mammal.

30. A method for increasing the fecal excretion of cholesterol in a mammal, which comprises administering an effective cholesterol fecal excretion increasing amount of a compound according to any of claims 19 to 21 to the mammal.

31. A method for the prophylaxis or treatment of a clinical condition in a mammal, for which a cholesterol uptake inhibitor is indicated, which comprises administering a therapeutically effective amount of a compound according to any of claims 19 to 21 to the mammal.

32. A method for reducing the incidence of coronary heart disease-related events in a mammal, which comprises administering an effective coronary heart disease-related events reducing amount of a compound according to any of claims 19 to 21 to the mammal.

33. A method for reducing the concentration of cholesterol in the blood plasma or serum of a mammal, which comprises administering an effective cholesterol reducing amount of a compound according to any of claims 19 to 21 to the mammal.

34. A method for reducing blood plasma or serum concentrations of C-reactive protein (CRP) in a mammal, which comprises administering a therapeutically effective amount of a compound according to any of claims 19 to 21 to the mammal.

35. A method for reducing blood plasma or serum concentrations of triglycerides in a mammal, which comprises administering a therapeutically effective amount of a compound according to any of claims 19 to 21 to the mammal.

36. A method for increasing blood plasma or serum concentrations of HDL cholesterol of a mammal, which comprises administering a therapeutically effective amount of a compound according to any of claims 19 to 21 to the mammal.

37. A method for reducing blood plasma or serum concentrations of apolipoprotein B,in a mammal, which comprises administering a therapeutically effective amount of a compound according to any of claims 19 to 21 to the mammal.
